(19) United States
(12) Patent Application Publication
(10) Pub. No.: US 2025/0277215 A1
(43) Pub. Date: Sep. 4, 2025

(54) Long Non-Coding RNA 122 (Lnc122) for Treatment Cancer

(71) Applicant: The Board of Trustees of the Leland Stanford Junior University, Stanford, CA (US)

(72) Inventors: Mark A. Kay, Los Altos, CA (US); Hagoon Jang, Redwood City, CA (US)

(21) Appl. No.: 18/859,103

(22) PCT Filed: Apr. 25, 2023
(86) PCT No.: PCT/US2023/066201

§ 371 Date: Oct. 22, 2024

Related U.S. Application Data
(60) Provisional application No. 63/336,977, filed on Apr. 29, 2022.

(51) Int. Cl. C12N 15/113; A61K 38/46; A61P 35/00; C12N 15/86

(52) U.S. Cl. CPC C12N 15/1135; A61K 38/465; A61P 35/00; C12N 15/86; C12N 2750/14143

(57) ABSTRACT

Provided are methods and compositions for reducing proliferation of a target cell (e.g., target cancer cell), e.g., for treating an individual who has cancer. The subjects include the use of an agent that increases activity of long non-coding RNA 122 (lnc122) in the targeted cell(s). As such, a subject method can include a step of administering to the individual a subject composition, which includes an agent that increases activity of lnc122. In some cases, a subject agent is a genome targeting fusion protein (e.g., Zinc Finger, TALE, or CRISPR effector protein fused to a transcriptional activator) that causes increased transcription of lnc122 from its endogenous locus. In some cases, a subject agent is lnc122, or a functional equivalent thereof. In some cases, a subject agent is a nucleic acid that encodes lnc122 or a functional equivalent thereof.

### CROSS REFERENCE

This application claims benefit of U.S. Provisional Patent Application No. 63/336,977, filed Apr. 29, 2022, which application is incorporated herein by reference in its entirety.

### STATEMENT REGARDING FEDERALLY SPONSORED RESEARCH

This invention was made with Government support under contract AI071068 awarded by the National Institutes of Health. The Government has certain rights in the invention.

### INCORPORATION BY REFERENCE OF SEQUENCE LISTING PROVIDED AS AN XML FILE

A Sequence Listing is provided herewith as a Sequence Listing XML, “STAN-1972WO_SEQ_LIST.xml” created on Mar. 13, 2023 and having a size of 41,331 bytes. The contents of the Sequence Listing XML are incorporated by reference herein in their entirety.

### INTRODUCTION

micro RNAs (miRNAs) are generally classified in an intragenic or intergenic miRNA based on their genomic location. Intragenic miRNAs are subclassified as either intronic or lncRNA and a considerable number of these intragenic miRNAs are transcribed with their host genes to have functional linkage with it.

On the other hand, intergenic miRNAs have their own promoter and are independently transcribed. The primary transcripts of intergenic miRNAs have been considered not as an independent non-coding RNA but as an intermediate product for mature miRNAs. Long Non-coding RNAs (lncRNA, >200 nucleotides length) have been studied for decades (see, e.g., J. L. Rinn, H. Y. Chang, Annu Rev Biochem 89, 283-308 (2020)). A substantial number of lncRNAs share properties with mRNAs, e.g., Pol II mediated transcription, elongation, splicing, 5′ methylated guanosine, and polyadenylation of the 3′ end. It has been demonstrated that lncRNAs are involved in various biological functions such as epigenetic control, transcriptional regulation, miRNA sponging. and structural scaffolding. Emerging evidence have shown that the lnc-pri-miRNAs also have important biological roles in development and disease.

miR-122 is a liver specific intergenic miRNA whose portion is more than 70% in total hepatic miRNA pool. The function of miR-122 has been studied in liver lipid/cholesterol metabolism as well as liver tumorigenesis (see, e.g., Hsu et al., J Clin Invest 122, 2871-2883 (2012); Wen and Friedman, J Clin Invest 122, 2773-2776 (2012); and Esau et al., Cell Metab 3, 87-98 (2006)).

Lnc122 exists in both spliced and un-spliced form in human liver and is stably expressed in liver even after microprocessor processing.

### SUMMARY

The work described in the experimental examples below was initiated to study the biological function/role of lnc122 (also referred to herein as “lnc122 RNA”). This led to the surprising finding by the inventors that the absence of lnc122 RNA predisposed mice to high numbers of hepatocellular carcinomas (HCC), and the replacement of lnc122 RNA decreased the risk of HCC in mice predisposed to liver cancer or HCC.

Without wishing to be bound by theory, the activity of lnc122 appears to be due to the interaction of this long non-coding RNA with the UBR5 protein and the consequence this has on the oncogenic protein Myc. Myc deregulation occurs in about 30% of liver cancers and about 70% of total human cancer.

UBR5 is a ubiquitin ligase that selectively binds to the Myc oncogenic protein resulting in the degradation of Myc. The inventors have discovered that lnc122 is a mediator of UBR5-dependent Myc degradation and that lnc122 and Myc levels are negatively correlated. That is-Myc levels increase when lnc122 levels decrease, and Myc levels decrease when lnc122 levels increase. Likewise, reduced levels of lnc122, which results in increased Myc level, accelerates cancer development (e.g., liver cancer); while increased levels of lnc122, which results in decreased levels of Myc, can block cancer development (e.g., liver tumorigenesis).

Thus, the inventors have surprisingly discovered that enhancing expression of lnc122 in targeted cells (e.g., cancer cells) can be used as an anti-cancer therapy. For example, the data provided here demonstrate that lnc122 activation (i.e., increased levels of lnc122) blocks liver tumorigenesis.

The Myc oncogene is deregulated in approximately 30% of liver cancers and up to 70% of human cancers. While expression of lnc122 is naturally fairly liver specific, the UBR5 protein is ubiquitous. Thus, increasing lnc122 RNA can be used in a wide variety of cancers (e.g., any cancer in which Myc is deregulated)—not just liver cancer—because artificially increasing lnc122 levels in any cell with UBR5 should lead to a UBR5-mediated decrease in Myc levels.

Provided are methods and compositions for reducing proliferation of a target cell (e.g., target cancer cell). In some embodiments, a subject method includes introducing a subject composition, which includes an agent that increases activity of (e.g., efficiency of, level of) long non-coding RNA 122 (lnc122), into the target cell. In some cases, the target cancer cell is in vitro (e.g., cell in culture) and in some cases the target cancer cell is in vivo (e.g., a method of treatment). As such, in some embodiments a subject method is a method of treating an individual in need (e.g., an individual who has cancer). Such methods can include a step of administering to the individual a subject composition, which includes an agent that increases activity of lnc122 (e.g., efficiency of, level of lnc122).

In some cases, an agent that increases the level of lnc122 (thereby increasing lnc122 activity) is a genome targeting fusion protein (e.g., ZF (Zinc Finger), TALE (Transcription activator-like effector), or CRISPR effector protein fused to a transcriptional activator (e.g., “CRISPRa”)) that causes increased transcription of lnc122 from its endogenous locus. For example, in some cases the agent that increases the level of lnc122 is a “CRISPRa” agent, which would include a guide RNA plus a CRISPR effector protein (e.g., a class 2 effector protein such as a type II effector (e.g., Cas9), a type V effector (e.g., Cas12), a catalytically ‘dead’ version of the effector, e.g., dCas9, and the like) fused to a transcriptional activator. In some cases, the agent that increases the level of lnc122 is a ZF or TALE fused to a transcriptional activator. As will be understood by a skilled artisan, a genome targeting fusion protein will generally be targeted at or near the promoter of the targeted gene-in this case lnc122.

In some cases, an agent that increases the level of lnc122 (thereby increasing lnc122 activity) is lnc122 itself or a nucleic acid encoding it. For example, in some cases the agent includes lnc122. In some cases the agent includes human lnc122 (see SEQ ID NO: 5). In some cases the agent includes mouse lnc122 (see SEQ ID NO: 6). In some such cases, the lnc122 can be transcribed in vitro. In some cases, the lnc122 can be chemically synthesized. In some cases, a subject agent includes a nucleic acid (e.g., viral vector, plasmid DNA, minicircle DNA, doggybone DNA, and the like) that encodes lnc122 (see, e.g., SEQ ID NO: 1 [with intron] and SEQ ID NO: 2 [without intron] for human DNA sequence encoding lnc122; and SEQ ID NO: 3 for mouse DNA sequence encoding lnc122). In some cases, the nucleic acid encoding lnc122 is a viral vector (e.g., an AAV such as AAV8). In some cases, the agent is a viral particle (virion) that includes lnc122 and/or a nucleic acid encoding lnc122.

In any of the above scenarios of the preceding paragraph, a functional equivalent of lnc122 can be used. As such, in some cases an agent includes a functional equivalent of lnc122 (which can in some cases be transcribed in vitro or chemically synthesized). In some cases, an agent includes a nucleic acid (e.g., viral vector, plasmid DNA, minicircle DNA, doggybone DNA, and the like) that encodes the functional equivalent. In some cases, an agent includes a virion that includes the function equivalent or the nucleic acid encoding it.

In some cases, a functional equivalent includes a nucleotide sequence that does not have 100% identity with an lnc122 reference sequence of choice (e.g., the sequence set forth as SEQ ID NO: 5 [human] or 6 [mouse]), but does have 85% or more sequence identity (e.g., 90% or more, 95% or more, 98% or more, 99% or more, 99.5% or more sequence identity) with the reference sequence. In some cases, the functional equivalent is a fragment (a portion) of lnc122 that retains the intended function. In some embodiments, a functional equivalent retains the structural features of human and/or mouse lnc122-see the structures depicted in FIG. 17 and FIG. 18. For example, in some cases a functional equivalent will not have 100% identity to a reference lnc122 (e.g., human or mouse), but will retain structural features present in the reference lnc122.

In some cases, the functional equivalent is a fragment of lnc122, but does not have 100% identity with the corresponding portion of the desired lnc122 reference sequence (e.g., it can have 85% or more, 90% or more, 95% or more, 98% or more, 99% or more, or 99.5% or more sequence identity with the corresponding portion of the lnc122 reference sequence).

In some cases, an agent that increase the activity of (e.g., the level of, the efficiency of) lnc122 is a small molecule. Such a small molecule agent can in some cases interact with lnc122, thereby increasing its overall efficiency (activity per molecule). In some cases, a small molecule agent can increase the level of lnc122 (e.g., by stabilizing lnc122 or by increasing the amount of lnc122 production/transcription).

With regard to the types of cancers that can be treated (and also cancer cells that can be targeted)—any type of cancer is appropriate. In some cases, the cancer is liver cancer, lung cancer, ovarian cancer, prostate cancer, colorectal cancer, renal cancer, bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, gastric cancer, cancer of the central nervous system, skin cancer, head and neck cancer, or blood cancer. In some cases, because (without wishing to be bound by theory) increased lnc122 is thought to function at least in part by reducing Myc activity, the cancer is one that exhibits deregulation of Myc (i.e., increased Myc expression/Myc activity). For example, in some cases the cancer is liver cancer, bile duct cancer, bladder cancer, brain cancer, cancer of the nervous system, breast cancer, cervical cancer, colon cancer, esophagus cancer, head and neck cancer, renal cancer, cancer of the large intestine, leukemia, lymphoma, lung cancer, melanoma, skin cancer, ovarian cancer, endometrial cancer, pancreatic cancer, prostate cancer, sarcoma, stomach cancer, testicular cancer, or cancer of the uterus. In some cases, the cancer is a Myc-driven cancer. For example, in some cases, the cancer is liver cancer, breast cancer, colorectal cancer, pancreatic cancer, lung cancer, brain cancer, or prostate cancer. In some cases, the cancer is a liver cancer (e.g., hepatocellular carcinoma (HCC), intrahepatic cholangiocarcinoma, hepatoblastoma). In some cases, the cancer is liver cancer (e.g., HCC).

In some embodiments, a subject composition is administered locally (e.g., intratumoral injection). In some cases, a subject composition is administered systematically (e.g., subcutaneous, intravenous, and the like).

In some embodiments, a subject method includes administration of a second treatment (e.g., a second agent, a cancer therapy) in addition to a composition that includes the first agent. For example, in some cases, a genome editing fusion protein can be used in combination with a second treatment (e.g., a second agent, a cancer therapy). In some such cases, the genome editing fusion protein is a CRISPRa agent (e.g., a CRISPR effector protein such as Cas9 fused to a transcriptional activator). In some cases, the genome editing fusion protein is a ZF or TALE fusion protein (e.g., fused to a transcriptional activator). In some cases, an lnc122 RNA (or a functional equivalent thereof) or a nucleic acid encoding it can be used in combination with a second treatment (e.g., a second agent, a cancer therapy).

In some embodiments, a second treatment includes a second agent-and the second agent is a cancer targeting agent specifically binds a cancer cell antigen. In some cases, the second agent includes one or more selected from the group consisting of: Atezolizumab, Avastin (Bevacizumab), Bevacizumab, Cabometyx (Cabozantinib-S-Malate), Cabozantinib-S-Malate, Cyramza (Ramucirumab), Infigratinib Phosphate, Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab), and Truseltiq (Infigratinib Phosphate).

In some embodiments, a second treatment is a cancer therapy. For example, in some cases, the second treatment is chemotherapy (i.e., chemotherapy is administered to the individual). In some cases, the second treatment is radiotherapy (i.e., radiotherapy is administered to the individual).

Also provided are reagents, compositions, and kits/systems that find use in practicing the subject methods. As an illustrative example, provided are compositions that include an agent that increases the level of lnc122 in target cells (see, e.g., the discussion above). In some cases, such compositions are formulated for administration into an individual who has cancer.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1. Annotated schematic drawing human lnc122 locus (human Chromosome 18, NC_000018.10, mouse Chromosome 18, NC_000084.7). Schematic is from Dhir et al., NSMB, 2015. miR-122 is the most abundant miRNA in liver (70% of total miRNA pool). miR-122 is associated with liver lipid and cholesterol metabolisms.miR-122 levels are decreased in hepatocellular carcinoma (HCC). lnc122 is the primary transcript of miR-122. lnc122 is ˜5 kb in mouse and ˜2 kb in human. In human the lnc122 transcript is ˜5 kb prior to removal of an ˜3 kb intron via splicing.

FIG. 2. Results from proliferation experiments (assessed via MTS assay). Cell number of Ctrl Huh7 cells and lnc122−/miR 122+Huh7 cells were measured by an MTS assay on days 0, 1, 2, and 3. Cells deficient in lnc122 were more proliferative. When Huh7 were transduced with a vector resulting in 20× more lnc122 RNA than baseline, the cell lines were lost suggesting they were not able to proliferate with the over expression of lnc122.

FIG. 3. Human hepatoma cells (Huh7) made deficient in lnc122 had up regulation of Myc responsive genes. CRISPRi technology was used to reduce lnc122 expression and miR122, a microRNA that is processed from lnc122 precursor RNA, was added back. This showed that the effect was specific for lnc122 and not miR122. GSEA analysis were performed using RNAseq of ctrl Huh7 and lnc122−/miR122+Huh7 cells.

FIGS. 4A-4B. (FIG. 4A) Loss of lnc122 expression enhances MYC protein levels. Ctrl Huh7 cells and lnc122−/miR122+Huh7 cells were treated with cycloheximide and at various time points Myc protein levels were determined by western blot. Myc protein stability was increased in lnc122−/miR122+cells compared to ctrl Huh7 cells. (FIG. 4B) Expression of lnc122 decreased MYC protein levels. Ctrl Huh7 cells, lnc122−/miR122-Huh7 cells, lnc122−/miR122−/smlnc122+Huh7 cells and lnc122−/miR122−/pAlnc122+Huh7 cells were treated with cycloheximide. Myc protein stability decreased when smlnc122 and pAlnc122 overexpressed.

FIGS. 5A-5B. (FIG. 5A) Schematic depicting screening experiment to identify proteins that bound to lnc122. (FIG. 5B) lnc122 RNA-associated protein network in Huh7 cell revealed by Cytoscape. Proteins with known functions are annotated together. Color and value depicts the fold change of peptide counts of sense lnc122 group compared to antisense lnc122 group.

FIG. 6. Lnc122 binds to UBR5 protein, a Myc protein ubiquitin ligase. RNA immunoprecipitations were performed with the indicated antibodies in Huh7 cells. After immunoprecipitation, enriched RNA levels were measured by qRT-PCR.

FIG. 7. Ubr5 protein interacts with Myc protein. Co-immunoprecipitations were performed using myc antibody in ctrl and MG132 protcasome inhibitor treated Huh7 cells. IP, immunoprecipitation.

FIGS. 8A-8B. Lnc122 knockdown in mouse liver resulted in high levels of HCC tumors. (FIG. 8A) 6 weeks old FVB/N male mice were injected with AAV-crispri_SCR (negative control) and AAV-crispri_lnc122 (to decrease lnc122 levels) and 1 week later, the mice were injected with a a sleeping-beauty transposon mediating myc expression by hydrodynamic injection. Mice were sacrificed at 11 weeks old to assess liver tumorigenesis. (FIG. 8B) 6 weeks old FVB/N male mice were injected with AAV-crisprA_SCR (negative control) and AAV-crisprA_lnc122 (to increase lnc122 levels) and 1 week later, the mice were injected sleeping-beauty transposon mediating myc expression by hydrodynamic injection. Mice were sacrificed at 15 weeks old to assess liver tumorigenesis.

FIG. 9. HCC patient samples were obtained from Stanford Tissue Bank and miR122 and lnc122 levels were assessed-both were reduced in HCC compared to its paired normal liver.

FIG. 10. Lnc122 also binds to TEAD transcription factors that are part of the Hippo signaling pathway, which is also involved in oncogenesis. RNA immunoprecipitation were performed indicated antibodies in Huh7 cells. After immunoprecipitation, enriched RNA levels were measured by qRT-PCR.

FIG. 11. Lnc122 binding to TEAD transcription factors. RNA immunoprecipitation were performed indicated antibodies in Huh7 cells. After immunoprecipitation, enriched RNA levels were measured by qRT-PCR.

FIG. 12. The effect of lnc122 RNA on Tead responsive gene expression. Volcano plot of TEAD target genes in RNAseq of ctrl Huh7 and lnc122−/miR 122+Huh7 cell.

FIG. 13. Mouse lnc122 interacts with Tead transcription factors. RNA immunoprecipitations were performed using the indicated antibodies in Hepal-6 mouse hepatoma cells. After immunoprecipitation, enriched RNA levels were measured by qRT-PCR

FIG. 14. Schematic of the Ubiquitin mediated protein degradation pathway.

FIG. 15. Human DNA sequence encoding human lnc122. Brackets indicate intron that is removed via splicing.

FIG. 16. Mouse DNA sequence encoding mouse lnc122. The mouse sequence does not naturally include an intron.

FIG. 17. Structural folding prediction for human lnc122. Red boxes are to emphasize special and similar structures (when comparing human and mouse lnc122). Structures were generated using the RNAfold web server at “uni” followed by “vie.” followed by “ac.” followed by “at”.

FIG. 18. Structural folding prediction for mouse lnc122. Red boxes are to emphasize special and similar structures (when comparing human and mouse lnc122). Structures were generated using the RNAfold web server at “uni” followed by “vie.” followed by “ac.” followed by “at”.

FIG. 19. lnc122 overexpression in non-hepatic cell (HeLa cell) led to reduction in Myc levels. Plasmids which contain mock, wtlnc122(lnc122+/miR122+), smlnc122(lnc122+/miR122−), and pAlnc122(lnc122+/miR122−) were transfected by lipofectamine 3000 reagent (invitrogen). 72 hr after transfection, cells were harvested and Myc protein levels were determined by western blotting.

FIG. 20. Huh7 cells were treated with MG132 (20 mM) for 4 h. The cell lysates were subjected to immunoprecipitation with IgG, MYC antibody, and MYC antibody with RNaseT1 (25U/ml of lysates) overnight at 4°° C. Input and RNA samples were incubated with/without RNaseT1 overnight at 4° C.

FIG. 21. Control Huh7 cells with Mock plasmid transfection and lnc122−/miR-122+Huh7 cells with Mock and wtlnc122 plasmid transfection were treated with MG132 (20 mM) for 4 h. The cell lysates were subjected to immunoprecipitation with an anti-MYC antibody overnight at 4° C. IP, immunoprecipitation

FIGS. 22A-22C. (FIG. 22A) Images of CRISPRi_SCR and CRISPRi_lnc122+miR-122 injected liver. Scale bar, 1 cm (FIG. 22B) H&E images of CRISPRi_SCR and CRISPRi_lnc122+miR-122 injected liver. Scale bar, 100 mm (FIG. 22C) H&E staining based hepatic carcinoma area quantification of CRISPRi_SCR and CRISPRi_lnc122+miR-122 injected liver tumor.

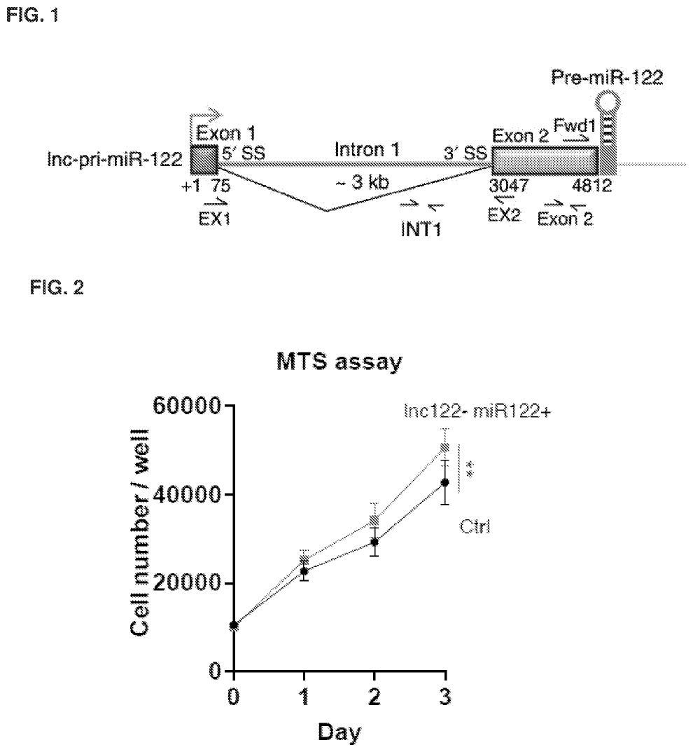

### DETAILED DESCRIPTION

As noted above, provided are methods and compositions for reducing proliferation of a target cell (e.g., target cancer cell). In some embodiments, a subject method includes introducing a subject composition, which includes an agent that increases the activity of (e.g., the efficiency of, the level of) long non-coding RNA 122 (lnc122), into the target cell. In some cases, the target cancer cell is in vitro (e.g., cell in culture) and in some cases the target cancer cell is in vivo (e.g., a method of treatment). As such, in some embodiments a subject method is a method of treating an individual in need (e.g., an individual who has cancer). Such methods can include a step of administering to the individual a subject composition, which includes an agent that increases activity of lnc122 (e.g., the efficiency of, the level of lnc122). In some case, the agent is a genome targeting fusion protein (e.g., ZF (Zinc Finger), TALE (Transcription activator-like effector), or CRISPR effector protein fused to a transcriptional activator (CRISPRa)) that causes increased transcription of lnc122 from its endogenous locus. In some cases, the agent is lnc122 (e.g., in some cases human lnc122, in some cases mouse lnc122), or a functional equivalent thereof. In some cases, the agent is a nucleic acid (e.g., plasmid, minicircle, viral vector, doggybone DNA, and the like) that encodes lnc122, e.g., in some cases human lnc122, in some cases mouse lnc122, or a functional equivalent thereof. In some cases the nucleic acid is an AAV vector such as an AAV8 vector.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges and/or values are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that as used herein and in the appended claims, the singular forms “a,” “an,” and “the” include plural referents unless the context clearly dictates otherwise. As such, the articles “a” and “an” arc used herein to refer to one or to more than one (i.e., to at least onc) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for usc of such exclusive terminology as “solely,” “only” and the like in connection with the recitation of claim elements, or use of a “negative” limitation.

It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Compositions and Methods

As summarized above, provided are methods and compositions for reducing proliferation of a target cell (e.g., target cancer cell). In some embodiments, a subject method includes introducing a subject composition, which includes an agent that increases activity of (e.g., efficiency of, level of) long non-coding RNA 122 (lnc122), into the target cell.

In some cases, a subject method is a method of reducing the proliferation rate of a target cell. In some case, proliferation rate will be reduced by 2% or more (i.e., after introduction of a subject agent, the cell proliferation rate is 98% or less what it would have been in the absence of said introduction). For example, in some cases, proliferation rate will be reduced by 3% or more (e.g., 4% or more, 5% or more, 6% or more, 7% or more, 8% or more, 9% or more, 10% or more, 15% or more, or 20% or more). In some cases the target cell is in vitro (e.g. a cell in culture). In some cases the target cell is in vivo (e.g., when a subject agent is administered to a subject as part of a method of treatment). As such, in some embodiments, a subject method is a method of treating an individual in need (e.g., an individual who has cancer). Such methods can include a step of administering to the individual a subject composition, which includes an agent that increases activity of lnc122 (e.g., efficiency of, level of lnc122).

i. Agents that Increase lnc122 Activity

An increase in lnc122 activity can manifest in a number of different ways. For example, lnc122 activity can increase due to an increased amount (level) of lnc122 present in a cell. As such, increasing the expression of lnc122 from its endogenous locus is one way to achieve increased lnc122 activity. Another way to achieve increased lnc122 activity by increasing the level of lnc122 in a cell is to directly provide lnc122 to the cell. Likewise, a nucleic acid encoding lnc122 can be provided to the cell, e.g., where the nucleotide sequence that encodes lnc122 is operably linked to a promoter (e.g., an endogenous promoter or a heterologous promoter as desired). Another way to achieve increased lnc122 activity by increasing the level of lnc122 is to stabilize lnc122 already present in the cell—e.g., by providing a small molecule that blocks the degradation of lnc122. Lnc122 activity can also be increased by increasing the efficiency of lnc122 molecules—e.g., by providing a small molecule that increases the functional efficiency of individual lnc122 molecules. It is to be noted that all of the above also apply to ‘equivalents thereof’ (such as when using the phrase ‘lnc122 or an equivalent thereof’).

In some cases, a subject agent increases lnc122 levels present in a target cell (e.g., a cancer cell) 1.2-fold or more (e.g., 1.5-fold or more, 2-fold or more, 2.5-fold or more, 3-fold or more, 5-fold or more, or 10-fold or more). Because it is thought that (without wishing to be bound by theory) increased lnc122 levels cause decreased Myc protein levels—in some cases a subject method (e.g., introducing into a cell and/or administering to an individual) includes a step of measuring Myc protein level in a target cell after a subject agent is introduced/administered. In some cases, a subject agent decreases Myc protein level present in a target cell (e.g,, cancer cell) to 90% or less (e.g., 85% or less, 80% or less, 70% or less, 60% or less, 50% or less, 40% or less, 30% or less, or 20% or less) what the Myc protein level would be if the agent was not introduced to the cell. In other words, in some cases, a subject agent decreases Myc protein level present in a target cell (e.g,, cancer cell) by 10% or more (thus resulting in the cell having 90% or less of the Myc levels it would otherwise have). For example, in some cases, a subject agent decreases Myc protein level present in a target cell (e.g,, cancer cell) by 15% or more (e.g., 20% or more, 30% or more, 40% or more, 50% or more, or 60% or more).

i(a). lnc122 and Nucleic Acids Encoding

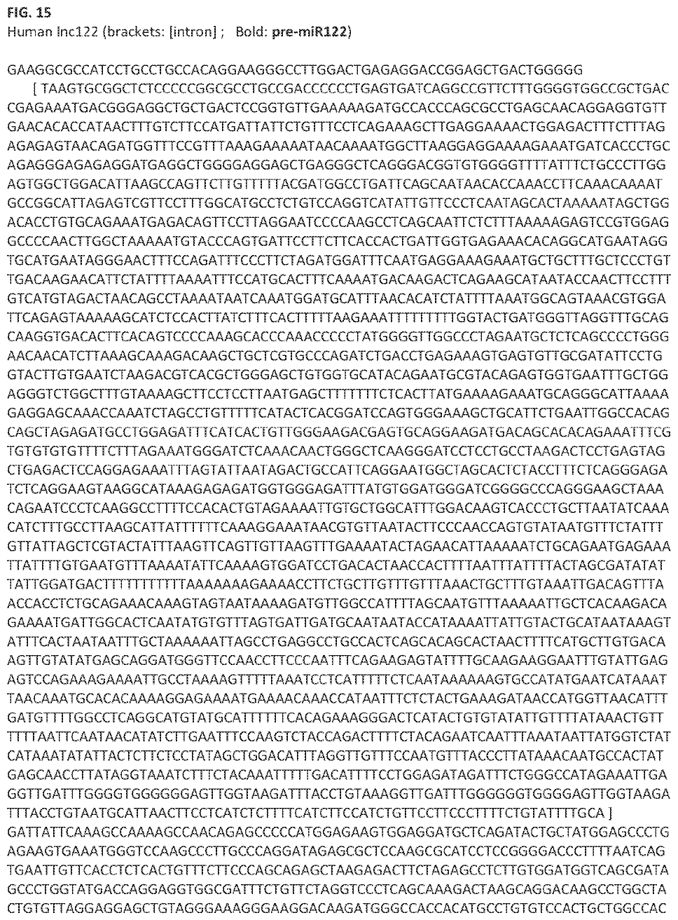

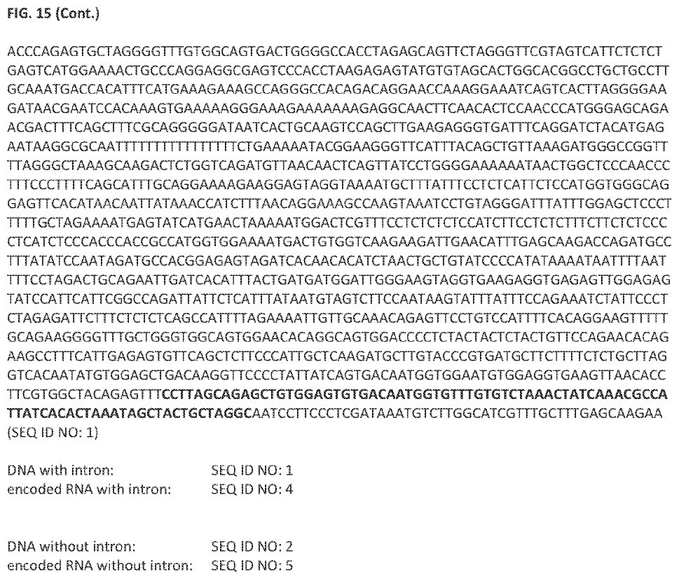

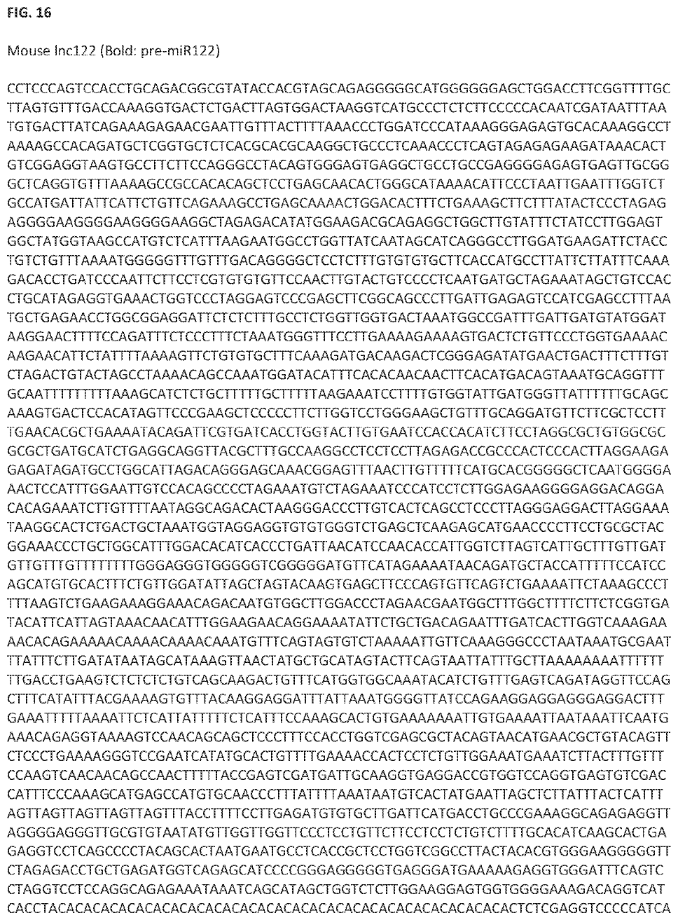

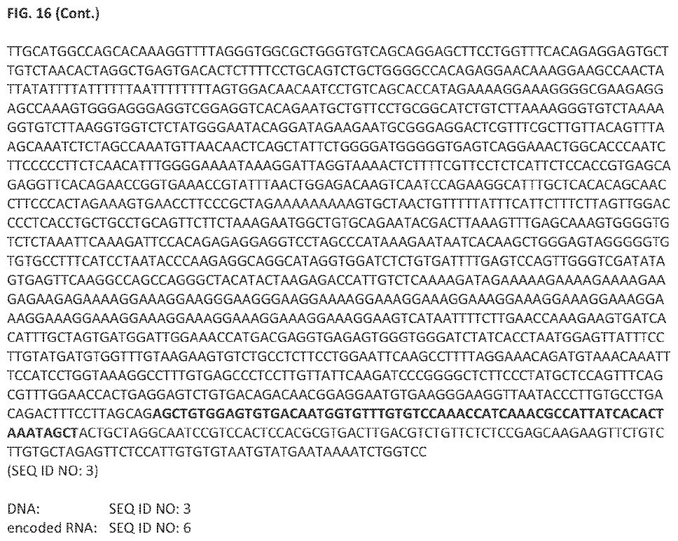

In some cases, an agent that increases the level of lnc122 (thereby increase lnc122 activity) is lnc122 itself or a nucleic acid encoding it. For example, lnc122 can be introduced into a cell as RNA. The sequence of human and mouse lnc122 are provided in FIG. 15 and FIG. 16. As can be understood from the figures, human lnc122 is naturally transcribed as an intron-containing precursor that undergoes splicing. The figures provide both intron-containing and intron-free (post splicing) sequences for human lnc122—mouse lnc122 is intron-less. RNA and DNA encoding the RNA are both provided in the figures.

In some cases the agent includes lnc122. In some cases the agent includes human lnc122 (SEQ ID NO: 5; see FIG. 15). In some cases, the agent includes mouse lnc122 (SEQ ID NO: 6; see FIG. 16). An lnc122 can be produced by any convenient method, e.g., by purifying it from cells, by direct chemical synthesis, by transcription in vitro from a DNA encoding the lnc122, and the like. Methods of synthesizing RNA from a DNA templates are well known in the art. In some cases, the lnc122 will be synthesized in vitro using an RNA polymerase enzyme (e.g., T7 polymerase, T3 polymerase, SP6 polymerase, etc.). Lnc122 and/or a nucleic acid encoding lnc122 can introduced into a cell by any convenient method for introducing nucleic acids into cells (e.g., microinjection, electroporation, transfection, transduction, e.g., using a virus, and the like)—and many such techniques will be available to the skilled artisan.

### Modified Nucleic Acids

A subject nucleic acid (e.g., an lnc122) can in some cases be modified.

### Modified Backbones/Internucleoside Linkages

Suitable nucleic acids containing modifications include nucleic acids containing modified backbones or non-natural internucleoside linkages. Nucleic acids (having modified backbones include those that retain a phosphorus atom in the backbone and those that do not have a phosphorus atom in the backbonc.

Suitable modified nucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable nucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid comprises one or more phosphorothioate and/or heteroatom internucleoside linkages, in particular —CH₂—NH—O—CH₂—, —CH₂—N(CH₃)—O—CH₂—(known as a methylene (methylimino) or MMI backbone), —CH₂—O—N(CH₃)—CH₂—, —CH₂—N(CH₃)—N (CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methylencimino and methylenchydrazino backbones; sulfonate and sulfonamide backbones; amide backbones; and others having mixed N, O, S and CH₂ component parts.

### Mimetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of nucleotides which are less likely to form undesired interactions with cellular proteins (Dwaine A. Braasch and David R. Corey, Biochemistry, 2002, 41 (14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and nucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene ( —CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

An “ethylene-bridged nucleic acid” (ENA) refers to an LNA modified RNA nucleotide where the ribose moiety is modified with an extra bridge containing two carbon atoms between the 2′ oxygen and the 4′ carbon (see, e.g., Morita et al., Bioorganic Medicinal Chemistry, 2003, 11 (10), 2211-2226). Ethylene-bridged nucleic acids are also encompassed by the term “bicyclic nucleic acids” or “bridged nucleic acids” (BNA).

A “constrained ethyl (cEt)” refers to an LNA modified RNA nucleotide where the ribose moiety is modified with an extra bridge connecting the 2′ oxygen and 4′ carbon, wherein the carbon atom of the bridge includes a methyl group. In some cases, the cEt is(S)-constrained ethyl. In other cases, the cEt is (R)-constrained ethyl (see, e.g., Pallan et al., Chem. Commun. (Camb)., 2012, 48 (66), 8195-8197). Constrained ethyl nucleic acids are also encompassed by the term “bicyclic nucleic acids” or “bridged nucleic acids” (BNA).

A “bicyclic nucleic acid” or a “bridged nucleic acid” (BNA) refers to a modified RNA nucleotide where the ribose moiety is modified with an extra bridge connecting the 2′ oxygen and 4′ carbon, thereby forming a bicyclic ring system. BNA monomers can contain a five-membered, six-membered or a seven-membered bridge structure with a fixed 3′-endo conformation. Bridged nucleic acids include without limitation, locked nucleic acids (LNA), ethylene-bridged nucleic acids (ENA) and constrained ethyl (cEt). A “bridge” refers to a chain of atoms or a valence bond connecting two bridgeheads, where a “bridgehead” is any skeletal atom of a ring system (e.g., the ribose ring system) which is bonded to three or more skeletal atoms (excluding hydrogen). In some embodiments, the bridge in a BNA has 7-12 ring members and 1-4 heteroatoms independently selected from nitrogen, oxygen, or sulfur. Unless otherwise specified, a BNA is optionally substituted with one or more substituents, e.g., including, but not limited to alkyl, substituted alkyl, alkoxy, substituted alkoxy, hydroxy, amino and halogen.

### Modified Sugar Moieties

A subject nucleic acid can also include one or more substituted sugar moieties. Suitable polynucleotides comprise a sugar substituent group selected from: OH; F; O—, S—, or N-alkyl; O—, S—, or N-alkenyl; O—, S- or N-alkynyl; or O-alkyl—O—alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m arc from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an nucleotide, or a group for improving the pharmacodynamic properties of an nucleotide, and other substituents having similar properties. A suitable modification includes 2′-methoxyethoxy (2′—O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃)₂.

Other suitable sugar substituent groups include methoxy (—O—CH₃), aminopropoxy (—OCH₂ CH₂ CH₂NH₂), allyl (—CH₂—CH═CH₂), —O—allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido (5,4-(b) (1,4)benzoxazin-2(3H)-one), carbazole cytidine (2H-pyrimido(4,5-b) indol-2-one), pyridoindole cytidine (H-pyrido (3′,2′4,5)pyrrolo (2,3-d)pyrimidin-2-one).

Heterocyclic base moieties may also include those in which the purine or pyrimidine base is replaced with other heterocycles, for example 7-dcaza-adeninc, 7-deazaguanosinc, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Conjugates

Another possible modification of a subject nucleic acid involves chemically linking to the polynucleotide one or more moieties or conjugates which enhance the activity, cellular distribution or cellular uptake of the nucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.\

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to a subject nucleic acid. Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9 (6): 489-96); an Drosophila Antennapedia protein transduction domain (Noguchi et al. (2003) Diabetes 52 (7): 1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 8); Transportan GWTLNSAGYLLGKINLKALAALAKKIL (SEQ ID NO:9); KALAWEAKLAKALAKALAKHLAKALAKALKCEA (SEQ ID NO:10); and RQIKIWFQNRRMKWKK (SEQ ID NO:11). Exemplary PTDs include but are not limited to, YGRKKRRQRRR (SEQ ID NO:7), RKKRRQRRR (SEQ ID NO:12); an arginine homopolymer of from 3 arginine residues to 50 arginine residues; Exemplary PTD domain amino acid sequences include, but are not limited to, any of the following: YGRKKRRQRRR (SEQ ID NO:7); RKKRRQRR (SEQ ID NO:13); YARAAARQARA (SEQ ID NO:14); THRLPRRRRRR (SEQ ID NO:15); and GGRRARRRRRR (SEQ ID NO:16). In some embodiments, the PTD is an activatable CPP (ACPP) (Aguilera et al. (2009) Integr Biol (Camb) June; 1 (5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Nucleic Acids Encoding

Lnc122 can also be provide to a cell by introducing a nucleic acid that encodes lnc122 into the cell As such, in some cases, a subject agent includes a nucleic acid (e.g., viral vector, plasmid DNA, minicircle DNA, doggybone DNA, and the like) that encodes lnc122. See, e.g., SEQ ID NO: 1 [with intron] and SEQ ID NO: 2 [without intron] for human DNA sequence encoding lnc122; and SEQ ID NO: 3 for mouse DNA sequence encoding lnc122. Any convenient type of nucleic acid can be used. In some cases, the nucleic acid will be one that integrates into the genome of the cell and in some cases, the nucleic acid does not integrate. For example, in some cases the nucleic acid is episomal. The nucleic acid can be a DNA plasmid (supercoiled, nicked or linearised), minicircle DNA (linear or supercoiled), DNA generated using an enzymatic DNA amplification platform e.g. doggybone DNA (where the final DNA used is in a closed ligated form or where it has been prepared (e.g restriction enzyme digestion) to have open cut ends), and the like.

Suitable nucleic acids comprising nucleotide sequence encoding lnc122 include expression vectors, where an expression vector that includes a nucleotide sequence encoding lnc122 is a recombinant expression vector. In some cases the nucleotide sequence encoding lnc122 is operably linked to a promoter. Examples of suitable nucleic acids include viral vectors and non-viral vectors, e.g., plasmids, minicircles, and the like. In some embodiments, the nucleic acid is a viral vector. A viral vector refers to a virus or viral chromosomal material into which a DNA encoding lnc122 can be included for transfer into a cell. Any convenient virus that facilitates nucleic acid delivery (e.g., deliver of lnc122 RNA or delivery of lnc122-encoding DNA) may be used as a viral vector in the subject methods and compositions.

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

As noted above as one non-limiting example, one virus/viral vector of interest is adeno-associated virus. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector”, or “rAAV vector” it is meant an AAV virus or AAV viral chromosomal material comprising a polynucleotide sequence not of AAV origin (i.e., a polynucleotide heterologous to AAV), typically a nucleic acid sequence of interest such as a sequence encoding lnc122. In some cases, the sequence of interest (e.g., lnc122) is operably linked to a promoter (e.g., a heterologous promoter).

In general, the nucleic acid sequence of interest is flanked by at least one, and generally by two AAV inverted terminal repeat sequences (ITRs). In some instances, the recombinant viral vector also comprises viral genes important for the packaging of the recombinant viral vector material. By “packaging” it is meant a series of intracellular events that result in the assembly and encapsidation of a viral particle, e.g. an AAV viral particle. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A “viral particle” or “virion” refers to a single unit of virus comprising a capsid encapsidating a virus-based polynucleotide, e.g. the viral genome (as in a wild type virus), or, e.g., the subject targeting vector (as in a recombinant virus). An “AAV viral particle” refers to a viral particle composed of at least one AAV capsid protein (typically by all of the capsid proteins of a wild-type AAV) and an encapsidated polynucleotide AAV vector. If the particle comprises a heterologous polynucleotide (i.e. a polynucleotide other than a wild-type AAV genome, such as a transgene to be delivered to a mammalian cell, e.g., a nucleotide sequence encoding lnc122), it is typically referred to as an “rAAV vector particle” or simply an “rAAV vector”. Thus, production of rAAV particle necessarily includes production of rAAV vector, as such a vector is contained within an rAAV particle.

In some cases, the nucleic acid encoding lnc122 is a viral vector (e.g., an AVV such as AAV8). In some cases, the agent is a viral particle (virion) that includes lnc122 and/or a nucleic acid encoding lnc122.

A variety of methods for generating AAV virions are known in the art. Generally, the methods involve inserting or transducing an AAV vector into a host cell capable of packaging the AAV vector into an AAV virion. Exemplary methods are described and referenced below; however, any method known to one of skill in the art can be employed to generate the AAV virions.

An AAV vector comprising a heterologous nucleic acid and used to generate an AAV virion can be constructed using any convenient method, including methods that are well known in the art. See, e.g., Koerber et al. (2009) Mol. Ther., 17:2088; Koerber et al. (2008) Mol Ther., 16:1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector can be introduced into a suitable host cell using known techniques, such as by transfection. A number of transfection techniques arc generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome-mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Suitable host cells for producing AAV virions include any species and/or type of cell that can be, or have been, used as recipients of a heterologous AAV DNA molecule, and can support the expression of required AAV production cofactors from helper viruses. Such host cells can include but are not limited to microorganisms, yeast cells, insect cells, and mammalian cells, that can be, or have been, used as recipients of a heterologous DNA molecule. The term includes the progeny of the original cell transfected. Thus, a “host cell” as used herein generally refers to a cell transfected with an exogenous DNA sequence. Cells from the stable human cell line, HEK293 (readily available through, e.g., the American Type Culture Collection under Accession Number ATCC CRL1573) can be used. The human cell line HEK293 is a human embryonic kidney cell line that has been transformed with adenovirus type-5 DNA fragments (Graham et al. (1977) J. Gen. Virol. 36:59), and expresses the adenoviral Ela and Elb genes (Aiello et al. (1979) Virology 94:460). The HEK293 cell line is readily transfected, and provides a convenient platform in which to produce AAV virions.

Methods of producing an AAV virion in insect cells are known in the art, and can be used to produce a subject AAV virion. See, e.g., U.S. Patent Publication No. 2009/0203071; U.S. Pat. No. 7,271,002; and Chen (2008) Mol. Ther. 16:924.

In some embodiments, the AAV virion or AAV vector is packaged into an infectious virion or virus particle, by any of the methods described herein or known in the art.

As noted above, a nucleic acid encoding Lnc122 can be a doggybone DNA (dbDNA) (Touchlight Genetics, London, UK). Doggybone DNA is a minimal, linear, double stranded and covalently closed DNA construct. It can encode long, complex, or unstable DNA sequences, eliminates bacterial sequences and has a strong expression profile. Doggybone DNA is produced using an in vitro process for the production of closed linear DNA. See, e.g., Karda et al., Gene Therapy volume 26, pages86-92 (2019); and Scott et al., Hum Vaccin Immunother. 2015;11 (8): 1972-82; as well as U.S. patents application Ser. Nos. 20/120,282283 and 20190083602 and U.S. Pat. No. 9,109,250; all of which are incorporated by reference for their teachings related to doggybone DNA.

A nucleotide sequence encoding lnc122 can be operably linked to a control element (such as a promoter-in which case the combination can be referred to as an expression cassette) in a manner permitting transcription, translation and/or expression in a cell. Expression control sequences include appropriate transcription initiation, termination, promoter and enhancer sequences; efficient RNA processing signals such as splicing and/or polyadenylation (polyA) signals; sequences that stabilize cytoplasmic mRNA; and the like. A great number of expression control sequences, including promoters selected from native, constitutive, inducible and/or tissue-specific, are known in the art.

In some embodiments, the promoter used is a Pol II promoter (e.g., a constitutive promoter, an inducible promoter, a tissue specific promoter). In some embodiments, the promoter used is a Pol III promoter (e.g., U6, H1, 7SK).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the beta-actin promoter, the phosphoglycerol kinase (PGK) promoter, and the EF1 promoter (e.g., Invitrogen). Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clonetech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, thc T7 polymerase promoter system (WO 98/10088); the ccdysonc insect promoter (No et al., (1996) Proc. Natl. Acad. Sci. USA, 93:3346-3351), the tetracycline-repressible system (Gossen et al., (1992) Proc. Natl. Acad. Sci. USA, 89:5547-5551), the tetracycline-inducible system (Gossen et al., (1995) Science, 268:1766-1769, see also Harvey et al., (1998) Curr. Opin. Chem. Biol., 2:512-518), the RU486-inducible system (Wang et al., (1997) Nat. Biotech., 15:239-243 and Wang et al., (1997) Gene Ther., 4:432-441) and the rapamycin-inducible system (Magari et al., (1997) J. Clin. Invest., 100:2865-2872). Other types of inducible promoters useful in this context are those regulated by a specific physiological state, e.g., temperature, acute phase, a particular differentiation state of the cell, or in replicating cells only.

In some cases a nucleotide sequence of interest is operably linked to a tissue-specific promoter. For instance, if expression in skeletal muscle is desired, a promoter active in muscle should be used. These include the promoters from genes encoding skeletal.beta.-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others.

i(b). Functional Equivalent Thereof

In any of the above scenarios of the preceding paragraphs (e.g., those referring to introducing lnc122 as RNA or as DNA, nucleic acids encoding lnc122, modified nucleic acids, etc.), a functional equivalent of lnc122 can be used. As such, those paragraphs are not expressly repeated here. Thus, in some embodiments the phrase “lnc122 or a functional equivalent thereof” is substituted for the phrase “lnc122” above and in some embodiments the phrase “functional equivalent of lnc122” is substituted for the phrase “lnc122” above. For example, in some cases, a subject agent is a functional equivalent of lnc122 (transcribed in vitro, chemically synthesized, and the like). In some cases, a subject is a nucleic acid (e.g., viral vector, plasmid DNA, minicircle DNA, doggybone DNA, and the like) that encodes an lnc122 functional equivalent. In some cases, a subject agent is a virion that includes an lnc122 functional equivalent or a nucleic acid encoding it.

In some cases, an lnc122 functional equivalent (e.g., retains the intended lnc122 function as described above, e.g., reducing cell proliferation, reducing Myc level, interacting with UBR5, and the like) includes a nucleotide sequence that does not have 100% identity with an lnc122 reference sequence of choice (e.g., the sequence set forth as SEQ ID NO: 5 [human] or 6[mouse]), but has 85% or more sequence identity (e.g., 90% or more, 95% or more, 98% or more, 99% or more, 99.5% or more sequence identity) with the reference sequence. In some cases, a functional equivalent has 85% or more sequence identity (e.g., 90% or more, 95% or more, 98% or more, 99% or more, 99.5% or more sequence identity) with SEQ ID NO: 5. For example, in some cases a functional equivalent has 90% or more sequence identity (e.g., 95% or more, 98% or more, 99% or more, 99.5% or more sequence identity) with SEQ ID NO: 5. In some cases a functional equivalent has 95% or more sequence identity (e.g., 98% or more, 99% or more, 99.5% or more sequence identity) with SEQ ID NO: 5. In some cases, a functional equivalent has 85% or more sequence identity (e.g., 90% or more, 95% or more, 98% or more, 99% or more, 99.5% or more sequence identity) with SEQ ID NO: 6. For example, in some cases a functional equivalent has 90% or more sequence identity (e.g., 95% or more, 98% or more, 99% or more, 99.5% or more sequence identity) with SEQ ID NO: 6. In some cases a functional equivalent has 95% or more sequence identity (e.g., 98% or more, 99% or more, 99.5% or more sequence identity) with SEQ ID NO: 6.

In some cases, the functional equivalent is a fragment (a portion) of lnc122 that retains the intended lnc122 function (as described above, e.g., reducing cell proliferation, reducing Myc level, interacting with UBR5, and the like). In some cases, the functional equivalent is a fragment of lnc122, but does not have 100% identity with the corresponding portion of the desired lnc122 reference sequence (e.g., it can have 85% or more, 90% or more, 95% or more, 98% or more, 99% or more, or 99.5% or more sequence identity with the corresponding portion of the lnc122 reference sequence, e.g., SEQ ID NO: 5 or 6). In some cases, such a functional equivalent has 90% or more (e.g.,95% or more, 98% or more, 99% or more, or 99.5% or more) sequence identity with the corresponding portion of the lnc122 sequence set forth as SEQ ID NO: 5. In some cases, such a functional equivalent has 95% or more (e.g., 98% or more, 99% or more, or 99.5% or more sequence identity with the corresponding portion of the lnc122 sequence set forth as SEQ ID NO: 5. In some cases, such a functional equivalent has 90% or more (e.g.,95% or more, 98% or more, 99% or more, or 99.5% or more) sequence identity with the corresponding portion of the lnc122 sequence set forth as SEQ ID NO: 6. In some cases, such a functional equivalent has 95% or more (e.g., 98% or more, 99% or more, or 99.5% or more sequence identity with the corresponding portion of the lnc122 sequence set forth as SEQ ID NO: 6.

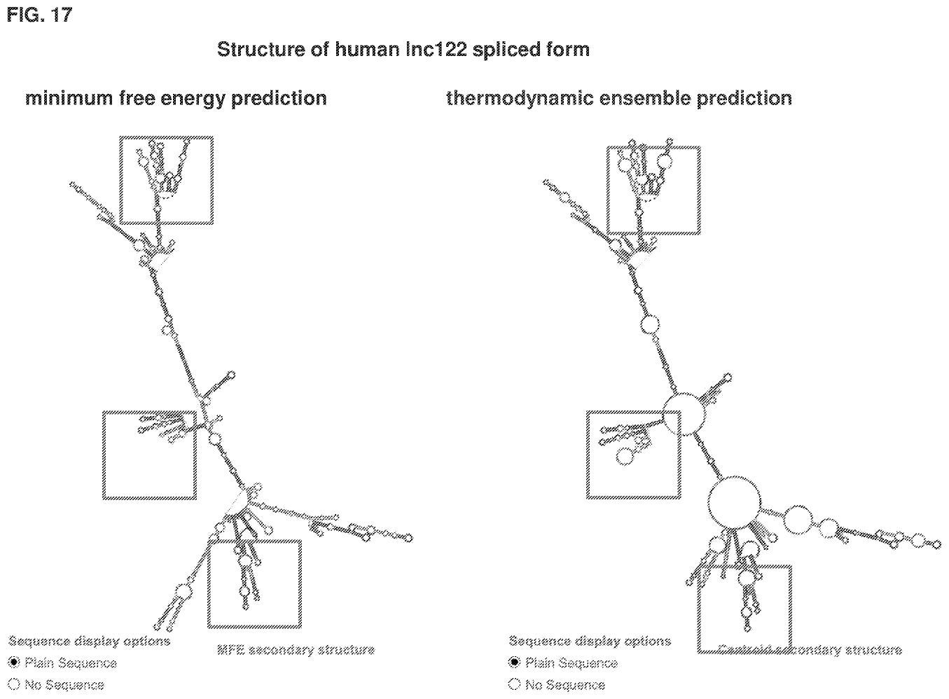

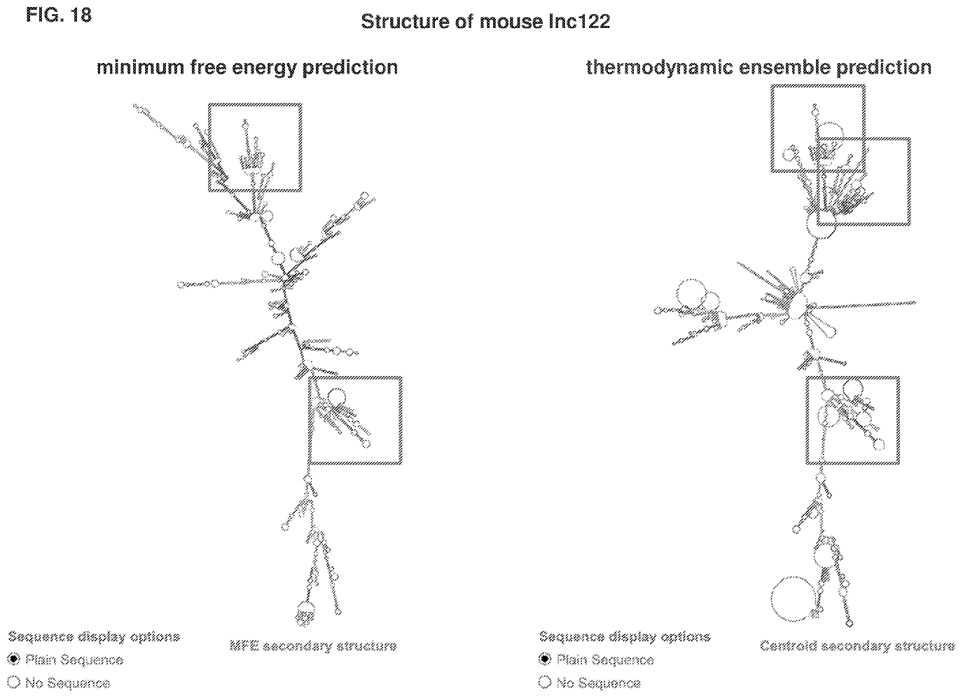

In some embodiments, a functional equivalent retains the structural features of human and/or mouse lnc122—see the structures depicted in FIG. 17 and FIG. 18. For example, in some cases a functional equivalent will not have 100% identity to a reference lnc122 (e.g., human or mouse), but will retain structural features present in the reference lnc122 (see the figures depicted in FIG. 17 and FIG. 18). Structural predictions were generated for human and mouse lnc122 (using the RNAfold web server at “uni” followed by “vie.” followed by “ac.” followed by “at”) and are provided in FIG. 17 and FIG. 18. Such structures can be used for guidance if designing a functional equivalent is desired. For example, in some cases a functional equivalent will not have 100% identity to a reference lnc122 (e.g., human or mouse), but will retain structural features present in the reference lnc122 (e.g., as depicted in FIG. 17-FIG. 18). In some cases, a functional equivalent will not have 100% identity to a reference lnc122 (e.g., human or mouse), but has 85% or more sequence identity (e.g., 90% or more, 95% or more, 98% or more, 99% or more, 99.5% or more sequence identity) with the reference lnc122 and also retains the structural features present in the reference lnc122 (e.g., as depicted in FIG. 17-FIG. 18). In some cases, such a functional equivalent has 90% or more sequence identity (e.g., 95% or more, 98% or more, 99% or more, 99.5% or more sequence identity) with SEQ ID NO: 5 and also retains the structural features present in the reference lnc122 (e.g., as depicted in FIG. 17-FIG. 18). In some cases, such a functional equivalent has 95% or more sequence identity (e.g., 95% or more, 98% or more, 99% or more, 99.5% or more sequence identity) with SEQ ID NO: 5 and also retains the structural features present in the reference lnc122 (e.g., as depicted in FIG. 17-FIG. 18). In some cases, such a functional equivalent has 90% or more sequence identity (e.g., 95% or more, 98% or more, 99% or more, 99.5% or more sequence identity) with SEQ ID NO: 6 and also retains the structural features present in the reference lnc122 (e.g., as depicted in FIG. 17-FIG. 18). In some cases, such a functional equivalent has 95% or more sequence identity (e.g., 95% or more, 98% or more, 99% or more, 99.5% or more sequence identity) with SEQ ID NO: 6and also retains the structural features present in the reference lnc122 (e.g., as depicted in FIG. 17-FIG. 18).

In some cases, a functional equivalent will be a fragment of (a portion of) an lnc122 (human or mouse), and that fragment will retain the structural features depicted in FIG. 17-FIG. 18. In some cases, a functional equivalent will be a fragment of (a portion of) an lnc122 (human or mouse), that fragment will retain the structural features depicted in FIG. 17-FIG. 18, and the fragment will not have 100% identity to the corresponding region of a reference lnc122 (e.g., human or mouse) but will have 85% or more sequence identity (e.g., 90% or more, 95% or more, 98% or more, 99% or more, 99.5% or more sequence identity) with the corresponding region of a reference lnc122 (e.g., human or mouse). In some cases, such a fragment has 90% or more sequence identity (e.g., 95% or more, 98% or more, 99% or more, 99.5% or more sequence identity) with the corresponding region of the lnc122 sequence set forth as SEQ ID NO: 5. In some cases, such a fragment has 95% or more sequence identity (e.g., 98% or more, 99% or more, 99.5% or more sequence identity) with the corresponding region of the lnc122 sequence set forth as SEQ ID NO: 5. In some cases, such a fragment has 90% or more sequence identity (e.g., 95% or more, 98% or more, 99% or more, 99.5% or more sequence identity) with the corresponding region of the lnc122 sequence set forth as SEQ ID NO: 6. In some cases, such a fragment has 95% or more sequence identity (e.g., 98% or more, 99% or more, 99.5% or more sequence identity) with the corresponding region of the lnc122 sequence set forth as SEQ ID NO: 6.

i(c). Genome Targeting Fusion Proteins

In some cases, an agent that increases the level of lnc122 is a genome targeting fusion protein (e.g., ZF (Zinc Finger), TALE (Transcription activator-like effector), or CRISPR effector protein fused to a transcriptional activator (CRISPRa)) that causes increased transcription of lnc122 from its endogenous locus. When referring to genome targeting fusion proteins, it is to be understood that they can be provided to cells in protein form or nucleic acid form (e.g., RNA or DNA encoding the protein). Genome targeting fusion proteins, the associated RNA (e.g., guide RNA), and/or nucleic acids encoding them can introduced into a cell by any convenient method for introducing nucleic acids into cells (e.g., microinjection, electroporation, transfection, transduction, e.g., using a virus, and the like)—and many such techniques will be available to the skilled artisan.

In some cases the agent that increases the level of lnc122 is a “CRISPRa” agent. As would be understood in the art, use of a CRISPRa agent would include use of a CRISPR guide RNA in addition to the CRISPR effector protein (e.g., dCas9) fused to a transcriptional activator-the guide RNA providing guidance to a particular target sequence of choice. In some cases, the agent that increases the level of lnc122 is a ZF or TALE fused to a transcriptional activator. As will be understood by a skilled artisan, a genome targeting fusion protein will generally be targeted at or near the promoter of the targeted gene—in this case lnc122. Any convenient transcriptional activator (or combination thereof) can be used for fusion to a genome-targeting protein—examples include but are not limited to VPH, VP16, VP48, VP64, VP160, VP192, VPR (VP64-p65-Rta), and the like.

If using a CRISPRa agent, in some cases a modified guide RNA can be used (sometimes referred to as a scaffold RNA (scRNA)). The modified guide RNA can include a protcin-binding region (such as an MS2, PP7, or com aptamer sequence) in order to recruit a transcriptional activator fused to an RNA-binding protein (e.g., MCP, PCP, Com). In this way, multiple activators can simultaneously be recruited to influence transcription. As a non-limiting example, the working examples below include use of a CRISPRa system that included: (1) a dCas9-VP64 fusion protein (a CRISPR effector protein (e.g., dCas9) fused to a transcriptional activator); (2) a modified guide RNA (having an MS2 hairpin); and (3) an MS2 coat protein fused to the p65subunit of NF-kappaB and the activation domain of human heat-shock factor 1 (HSF1). The guide RNA contained two aptamers, each capable of binding two MS2 coactivator proteins, effectively recruiting four coactivators for every CRISPR targeting activator complex. The CRISPR protein can also be fused to a GCN4 peptide array that attracts scFv-linked pro-activation domains (VP64, p65-HSF1, p300, and others).

For further details on various CRISPRa systems, also see, e.g., Brezgin et al., Int J Mol Sci. 2019 December; 20(23):6041. For additional information related to suitable genome targeting proteins and their guide nucleic acids (e.g., CRISPR/Cas effector proteins such as Cas9, CasX, CasY, and Cpf1/Cas12, Zinc finger (ZF) proteins, TALE proteins, CRISPR/Cas guide RNAs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192(2012); Zhang et al., Nat Biotechnol. 2011February;29(2):149-53; Zetsche et al, Cell. 2015 Oct. 22;163(3):759-71; Makarova et al, Nat Rev Microbiol. 2015 November;13(11):722-36; Shmakov et al., Mol Cell. 2015 Nov. 5;60(3):385-97; Jinek et al., Science. 2012 Aug. 17;337(6096):816-21; Chylinski et al., RNA Biol. 2013May; 10(5):726-37; Ma et al., Biomed Res Int. 2013;2013:270805; Hou et al., Proc Natl Acad Sci USA. 2013 Sep. 24;110(39):15644-9; Jinek et al., Elife. 2013;2: e00471; Pattanayak et al., Nat Biotechnol. 2013 September;31(9):839-43; Qi et al, Cell. 2013 Feb. 28;152(5):1173-83; Wang et al., Cell. 2013 May 9;153(4):910-8; Auer et. al., Genome Res. 2013 Oct. 31; Chen et. al., Nucleic Acids Res. 2013 Nov. 1;41(20): e19; Cheng et. al., Cell Res. 2013 October;23(10):1163-71; Cho et. al., Genetics. 2013 November;195(3):1177-80; DiCarlo et al., Nucleic Acids Res. 2013April;41(7):4336-43; Dickinson et. al., Nat Methods. 2013 October;10(10):1028-34; Ebina et. al., Sci Rep. 2013;3:2510; Fujii et. al, Nucleic Acids Res. 2013 Nov. 1;41(20): e187; Hu et. al., Cell Res. 2013 November;23(11):1322-5; Jiang et. al., Nucleic Acids Res. 2013 Nov. 1;41(20): e188; Larson et. al., Nat Protoc. 2013 November;8(11):2180-96; Mali et. at., Nat Methods. 2013 October;10(10):957-63; Nakayama et. al., Genesis. 2013 December;51(12):835-43; Ran et. al., Nat Protoc. 2013November;8(11):2281-308; Ran et. al., Cell. 2013 Sep. 12;154(6):1380-9; Upadhyay et. al., G3(Bethesda). 2013 Dec. 9;3(12):2233-8; Walsh et. al., Proc Natl Acad Sci U S A. 2013 Sep. 24;110(39):15514-5; Xie et. al., Mol Plant. 2013 Oct. 9; Yang et. al., Cell. 2013 Sep. 12;154(6):1370-9; Briner et al., Mol Cell. 2014 Oct. 23;56(2):333-9; Burstein et al., Nature. 2016 Dec. 22-Epub ahead of print; Gao et al., Nat Biotechnol. 2016 Jul 34(7):768-73; Koonin et al., Curr Opin Microbiol. 2017 June;37:67-78; Makarova et al., Nat Rev Microbiol. 2020February;18(2):67-83; and Shmakov et al., Nat Rev Microbiol. 2017 March;15(3):169-182; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

ii. Individuals and Target Cells

Provided are methods and compositions for reducing proliferation of a target cell (e.g., target cancer cell), and in some embodiments a subject method is a method of treating an individual in need (e.g., an individual who has cancer). Such methods can include a step of introducing into a cell or administering to an individual a subject composition, which includes an agent that increases activity of lnc122 (e.g., efficiency of, level of lnc122). The cancer (e.g., the type of cancer cell or the type of cancer that the individual has) can be any cancer.

As used herein “cancer” includes any form of cancer, e.g., solid tumor cancers (e.g., liver, lung, prostate, breast, bladder, colon, ovarian, pancreas, kidney, glioblastoma, medulloblastoma, leiomyosarcoma, head & neck squamous cell carcinomas, melanomas; etc.), leukemia, acute myeloid leukemia (AML), acute lymphoblastic leukemia (ALL), lymphomas, mesothelioma (MSTO), including both primary and metastatic tumors, and the like. In some cases, the individual has recently undergone treatment for cancer (e.g., radiation therapy, chemotherapy, surgical resection, etc.) and are therefore at risk for recurrence. Any and all cancers are suitable cancers to be treated/targeted by the subject methods, compositions, and kits. In some cases the cancer is liver cancer, bile duct cancer, bladder cancer, brain cancer, cancer of the nervous system, breast cancer, cervical cancer, colon cancer, esophagus cancer, head and neck cancer, renal cancer, cancer of the large intestine, leukemia, lymphoma, lung cancer, melanoma, skin cancer, ovarian cancer, endometrial cancer, pancreatic cancer, prostate cancer, sarcoma, stomach cancer, testicular cancer, or cancer of the uterus. In some cases, the cancer is liver cancer, lung cancer, ovarian cancer, prostate cancer, colorectal cancer, renal cancer, bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, gastric cancer, cancer of the central nervous system, skin cancer, head and neck cancer, or blood cancer.

In some cases, because (without wishing to be bound by theory) increased lnc122 is thought to function at least in part by reducing Myc activity, the cancer is one that exhibits deregulation of Myc (i.e., increased Myc expression/Myc activity). The ubiquity of MYC deregulation in cancer makes it an attractive therapeutic target with broad clinical potential. Deregulation of the MYC oncogene produces MYC protein that regulates almost every aspect of cancer cell metabolism, contributing to the acquisition of building blocks essential for cancer cell growth and proliferation. A large body of evidence has demonstrated that enhanced MYC expression is a major driving force of malignant transformation, and that both MYC-driven tumors and tumors driven by other oncogenes (e.g., KRAS) sustainedly depend on elevated MYC levels for growth. The major downstream effectors of MYC include those involved in ribosome biogenesis, mitochondrial biogenesis, protein translation, cell cycle progression, and metabolism. Accumulative evidence has shown that MYC plays an essential role in the regulation of global metabolic reprogramming, enabling rapid generation of bioenergetic substrates, and building blocks to sustain the uncontrolled cancer cell proliferation.

Myc deregulated cancers affect tissues such as: liver, bile duct, bladder, brain/nervous system, breast, cervix, colon, esophagus, head and neck, kidney/adrenal gland, large intestine, leukemia/lymphoma, lung, melanoma/skin, ovarian and endometrial, pancreas, prostate, sarcoma, stomach, testes, and uterus (see, e.g., Kalkat et al, Genes (Basel). 2017 June; 8(6):151; Dong et al., Signal Transduct Target Ther. 2020 Jul. 10;5(1):124; Madden et al., Mol Cancer. 2021 Jan. 4;20(1):3; and Wang et al., Signal Transduct Target Ther. 2021 Mar. 10;6(1):117). As such, in some cases the cancer is liver cancer, bile duct cancer, bladder cancer, brain cancer, cancer of the nervous system, breast cancer, cervical cancer, colon cancer, esophagus cancer, head and neck cancer, renal cancer, cancer of the large intestine, leukemia, lymphoma, lung cancer, melanoma, skin cancer, ovarian cancer, endometrial cancer, pancreatic cancer, prostate cancer, sarcoma, stomach cancer, testicular cancer, or cancer of the uterus.

In some cases, the caner is a Myc-driven cancer (i.e., a cancer for which Myc deregulation is the primary driver). For example, in some cases, the cancer is liver cancer, breast cancer, colorectal cancer, pancreatic cancer, lung cancer, brain cancer, or prostate cancer. In some cases, the cancer is a liver cancer (e.g., hepatocellular carcinoma (HCC), intrahepatic cholangiocarcinoma, hepatoblastoma). In some cases, the cancer is liver cancer (e.g., hepatocellular carcinoma (HCC), intrahepatic cholangiocarcinoma, hepatoblastoma). In some cases, the cancer is HCC.

In some embodiments, an individual to be treated is susceptible to, or is suspected of having an increased risk of acquiring (suspected of being susceptible to) cancer. In some such cases, the individual to be treated is susceptible to, or is suspected of having an increased risk of acquiring (suspected of being susceptible to) a Myc-deregulated cancer, a Myc-driven cancer, or a liver cancer (e.g., HCC). In some cases, the individual to be treated is susceptible to, or is suspected of having an increased risk of acquiring (suspected of being susceptible to) HCC.

The terms “cancer,” “neoplasm,” and “tumor” are used herein to refer to cells which exhibit autonomous, unregulated growth, such that they exhibit an aberrant growth phenotype characterized by a significant loss of control over cell proliferation. Cells of interest for detection, analysis, and/or treatment in the present disclosure include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell (e.g., from any of the cancers for which an individual can be treated, e.g., isolated from an individual having cancer) or is derived from a cancer cell e.g. clone of a cancer cell. For example, a cancer cell can be from an established cancer cell line, can be a primary cell isolated from an individual with cancer, can be a progeny cell from a primary cell isolated from an individual with cancer, and the like. In some cases, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, myelomas, etc., and circulating cancers such as leukemias.

In some cases a subject cancer cell is a liver cell. In some cases a subject cancer cell is a cell of (or from) a liver cancer.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma;

tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Sternberg cell. There are currently 6 recognized types of HL. Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) coursc. There are currently 61 recognized types of NHL. Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

iii. Treatment/Delivery/Administration

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the discasc. The term “treatment” encompasses any treatment of a discasc in a mammal, particularly a human, and includes: (a) preventing the disease and/or symptom(s) from occurring in a subject who may be predisposed to the disease or symptom(s) but has not yet been diagnosed as having it (prophylactic); (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with pre-cancerous tumors, lesions; those suspected of having cancer; etc.).

The terms “recipient”, “individual”, “subject”, “host”, and “patient”, are used interchangeably herein and refer to any mammalian subject for whom diagnosis, treatment, or therapy is desired (e.g., humans). This includes but is not limited to human and non-human primates (simians, apes, gibbons, gorillas, chimpanzees, orangutans, macaques, and the like); mammalian sport animals (e.g., horses); mammalian farm animals (e.g., poultry such as chickens and ducks, horses, cows, goats, sheep, pigs, etc.); mammalian pets (dogs, cats, etc.); and rodents/experimental animals (e.g., mouse, rat, rabbit, guinea pig, etc.) for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human. In some embodiments, the mammal is a rodent (e.g., rat, mouse). In some embodiments, the mammal is a non-human primate. Human subjects include fetal, neonatal, infant, juvenile and adult subjects. Subjects include animal disease models, for example, mouse and other animal models of cancer and others known to those of skill in the art.

A therapeutic treatment is one in which the subject is inflicted prior to administration and a prophylactic treatment is one in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to cancer and/or a family history indicating increased risk of cancer), in which case the treatment can be a prophylactic treatment.

A subject agent (one that increases lnc122 activity) can be administered by any suitable means (e.g., systemic or local). For example, a subject agent can be delivered systemically (e.g., intravenous, oral, subcutaneous), locally (e.g., local injection), or by any route, for example, by injection, infusion, orally (e.g., ingestion or inhalation), or topically (e.g., transdermally). A subject agent can be administered in any manner which is medically acceptable, including topical, oral, parenteral, intravenous, intracranial, intratumoral, intrapulmonary, and intranasal. Parenteral infusions include intramuscular, intravenous (bollus or slow drip), intraarterial, intraperitoneal, intrathecal or subcutaneous administration. This may include by injection (e.g., by parenteral routes such as intravenous, intravascular, intraarterial, subcutaneous, intramuscular, intratumoral, intraperitoneal, intraventricular, intracranial, or intraepidural), or others as well as oral, nasal, ophthalmic, rectal, or topical. Possible delivery and administration methods can include parenteral, intravenous, intramuscular, intraperitoneal, intradermal, subcutaneous, intracavity, intracranial, transdermal (topical), transmucosal and rectal administration. Example administration and delivery routes include intravenous, intraperitoneal, intrarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous, subcutaneous, oral). Sustained release administration is also specifically included in the disclosure, by such means as depot injections or erodible implants. Some agents can also applied directly to the area after a tumor is resected, e.g., by local injection, or by placing drug infused patties. In some cases a subject agent will be delivered systemically (e.g., intravenous, oral, subcutaneous, etc). In some cases a subject agent will be delivered locally (e.g., direct injection such as into a tumor, i.e., intratumoral injection).

A subject agent (one that increases lnc122 activity) can be formulated with a pharmaceutically acceptable carrier (one or more organic or inorganic ingredients, natural or synthetic, with which a subject agent is combined to facilitate its application). A suitable carrier can include sterile saline although other aqueous and non-aqueous isotonic sterile solutions and sterile suspensions known to be pharmaceutically acceptable are known to those of ordinary skill in the art.

Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the likc. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Anscl et al., cds., 7.sup.th ed., Lippincott, Williams, & Wilkins; and Handbook of Pharmaceutical Excipients (2000) A. H. Kibbe et al., eds., 3.sup.rd ed. Amer. Pharmaceutical Assoc.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. “treatSuch pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound. Preparations are typically sterilc and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Useful carriers include Vaseline.RTM., lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for subject agents and methods and uses of are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., lnc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured). In some cases, an effective amount is an amount that reduces tumor size (e.g., liver tumor size) in the individual. An effective amount can be determined on an individual basis and can be based, in part, on consideration of the symptoms to be treated and results sought. An effective amount can be determined by one of ordinary skill in the art employing such factors and using no more than routine experimentation.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease or disorder (e.g., liver cancer), one or more adverse symptoms, disorders, illnesses, pathologies, or complications, for example, caused by or associated with the discase, to a measurable extent, although decreasing, reducing, inhibiting, suppressing, limiting or controlling progression or worsening of the disease is also a satisfactory outcome.

A subject agent (one that increases lnc122 activity) can be administered as a pharmaceutical composition comprising an active therapeutic agent(s) and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered salinc, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear a subject agent (one that increases lnc122 activity) by non-covalent associations, such as non-covalent bonding or by encapsulation. The nature of the carrier can be either soluble or insoluble for purposes of the disclosure. Those skilled in the art will know of other suitable carriers for binding a subject agent (one that increases lnc122 activity), or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residucs) polypeptides; proteins, such as scrum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylatc) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of a subject agent (one that increases lnc122 activity) can be determined by standard pharmaceutical procedures in cell cultures and/or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition

A suitable subject agent (one that increases lnc122 activity) can be provided in pharmaceutical compositions suitable for therapeutic use, e.g. for human treatment. In some embodiments, pharmaceutical compositions of the present disclosure include one or more therapeutic entities of the present disclosure (e.g., one or subject agents) and can include a pharmaceutically acceptable carrier, a pharmaceutically acceptable salt, a pharmaceutically acceptable excipient, and/or esters or solvates thereof. In some embodiments, the use of a subject agent includes use in combination with (co-administration with) another therapeutic agent (e.g., another agent for preventing or treating cancer such as liver cancer, e.g., HCC). Therapeutic formulations comprising a subject agent can be prepared by mixing the agent(s) having the desired degree of purity with a physiologically acceptable carrier, a pharmaceutically acceptable salt, an excipient, and/or a stabilizer (Remington's Pharmaceutical Sciences 16th edition, Osol, A. Ed. (1980)) (e.g., in the form of lyophilized formulations or aqueous solutions). A composition having a subject agent can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane-and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this disclosure can be present in unsalified or unesterified form, or in salified and/or esterified form, and the naming of such compounds is intended to include both the original (unsalified and unesterified) compound and its pharmaceutically acceptable salts and esters.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to bc achieved, and (b) the limitations inherent in the art of compounding such active compound(s).

A “therapeutically effective dose” or “therapeutically effective amount” or “therapeutic dose” is an amount sufficient to elicit the intended biological, physiologic, clinical or medical response of a cell, tissue, organ, system, or subject that is being sought by the researcher, veterinarian, medical doctor or other clinician, e.g., reduced tumor size, stabilization of tumor size (e.g., prevention of increased tumor size), reduction or stabilization in the number of cancer cells present in an individual, prevention of metastasis, and the like. A therapeutically effective dose can be administered in one or more administrations. For purposes of this disclosure, a therapeutically effective dose of a subject agent (one that increases lnc122 activity) can be an amount that is sufficient to palliate, ameliorate, stabilize, reverse, prevent, slow or delay the progression of the disease state (e.g., liver cancer). Thus, in some cases, a therapeutically effective dose of a subject agent (one that increases lnc122 activity) reduces the size of a tumor (e.g., liver tumor). In some cases, a therapeutically effective dose of a subject agent (one that increases lnc122 activity) stabilizes the size of a tumor (e.g., liver tumor). In some cases, a therapeutically effective dose of a subject agent (one that increases lnc122 activity) reduces or stabilized the growth rate of a tumor (e.g., liver tumor). In some cases, a therapeutically effective dose of a subject agent (one that increases lnc122 activity) increases the life span of the individual being treated. In some cases, a therapeutically effective dose of a subject agent (one that increases lnc122 activity) improves the quality of life for the individual being treated. In some case, treatment using a subject method results in long term regression of the cancer such as liver cancer (e.g., increases the chance of survival of the individual being treated).

A subject agent (one that increases lnc122 activity) can be administered in any convenient amount using any convenient dosing regimen. The dose required to achieve a desired result can be proportional to the amount of time between doses and inversely proportional to the number of doses administered. Thus, as the frequency of dosing increases, the required dose decreases. The optimization of dosing strategies will be readily understood and practiced by one of ordinary skill in the art. Dosage and frequency may vary depending on the half-life of the agent in the patient. It will be understood by one of skill in the art that such guidelines will be adjusted for the molecular weight of the active agent. The dosage may also be varied for localized administration, e.g. intracranial, or for systemic administration, e.g. i.m., i.p., i.v., and the like.

### Co-Administration

In some embodiments, a subject method includes administration of a second treatment (e.g., a second agent, a cancer therapy) in addition to a composition that includes the first agent (which increases lnc122 activity). For example, in some cases, a genome editing fusion protein can be used in combination with a second treatment (e.g., a second agent, a cancer therapy). In some such cases, the genome editing fusion protein is a CRISPRa agent (e.g., a CRISPR effector protein such as Cas9 fused to a transcriptional activator). In some cases, the genome editing fusion protein is a ZF or TALE fusion protein (e.g., fused to a transcriptional activator). In some cases, an lnc122 (or a functional equivalent thereof) or a nucleic acid encoding it can be used in combination with a second treatment (e.g., a second agent, a cancer therapy).

The terms “co-administration” and “in combination with” include the administration of two or more therapeutic agents either simultaneously, concurrently or sequentially within no specific time limits. In some embodiments, the agents are present in the cell or in the subject's body at the same time or exert their biological or therapeutic effect at the same time. In some embodiments, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Treatment with a subject agent (e.g., which increases lnc122 activity) can be combined with another therapy such as chemotherapy, radiotherapy, and/or other therapies such as immunotherapies to enhance effect. In some embodiments, a second treatment includes a second agent—and the second agent is a cancer targeting agent specifically binds a cancer cell antigen.

In some cases, the second agent includes one or more selected from the group consisting of: Atezolizumab, Avastin (Bevacizumab), Bevacizumab, Cabometyx (Cabozantinib-S-Malate), Cabozantinib-S-Malate, Cyramza (Ramucirumab), Infigratinib Phosphate, Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab), and Truseltiq (Infigratinib Phosphate).

In some embodiments, a second treatment is a cancer therapy. For example, in some cases, the second treatment is chemotherapy (i.e., chemotherapy is administered to the individual). In some cases, the second treatment is radiotherapy (i.e., radiotherapy is administered to the individual).

Co-administration may involve concurrent (i.e. at the same time), prior, or subsequent administration of the drug/antibody with respect to the administration of an agent or agents of this disclosure. In some cases, a subject agent (one that increases lnc122 activity) is formulated with one or more agents that potentiate activity, or that otherwise increase the therapeutic effect (such as an immunomodulatory agent, a tumor-directed antibody, and the like).

In some cases, a subject agent (one that increases lnc122 activity) is used in a combination therapy (is co-administered) with a cancer targeting agent (e.g., an agent that specifically binds a cancer antigen, e.g., a cell-specific antibody selective for a tumor cell marker). Any convenient cancer cell targeting agent can be used. In some cases, the cancer cell targeting agent is a specific binding agent (e.g., a polypeptide such as an antibody that includes an antigen binding region specific for a cancer antigen) that specifically binds a cancer antigen of cancer cells (e.g., CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA). As such, in some cases, a subject method includes co-administering a subject agent (one that increases lnc122 activity) and a cancer cell targeting agent that is a specific binding agent (e.g., a polypeptide such as an antibody that includes an antigen binding region specific for a cancer antigen) that specifically binds an antigen (e.g., a cancer antigen) selected from: CD19, CD20, CD22, CD24, CD25, CD30, CD33, CD38, CD44, CD47, CD52, CD56, CD70, CD96, CD97, CD99, CD123, CD279 (PD-1), CD274 (PD-L1), EpCam, EGFR, 17-1A, HER2, CD117, C-Met, PTHR2, HAVCR2 (TIM3), and SIRPA.

In some cases, a subject agent (one that increases lnc122 activity) is used in a combination therapy (is co-administered) with: cetuximab (binds EGFR), panitumumab (binds EGFR), rituximab (binds CD20), trastuzumab (binds HER2), pertuzumab (binds HER2), alemtuzumab (binds CD52), brentuximab (binds CD30), tositumomab, ibritumomab, gemtuzumab, ibritumomab, or edrecolomab (binds 17-1A), or any combination thereof

In some cases, a subject agent (one that increases lnc122 activity) is used in a combination therapy (is co-administered) with a cancer drug such as: carboplatin, cisplatin, docetaxel (taxotere), gemcitabine (gemzar), nab-paclitaxel (abraxane), paclitaxel (taxol), pemetrexed (alimta), or vinorelbine (navelbine), or any combination thereof

In some cases, a subject agent (one that increases lnc122 activity) is used in a combination therapy (is co-administered) with a cancer drug such as: Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Capmatinib Hydrochloride, Carboplatin, Cemiplimab-rwlc, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gavreto (Pralsetinib), Gefitinib, Gilotrif (Afatinib Dimaleate), Gemcitabine Hydrochloride, Gemzar (Gemcitabine Hydrochloride), Imfinzi (Durvalumab), Infugem (Gemcitabine Hydrochloride), Ipilimumab, Iressa (Gefitinib), Keytruda (Pembrolizumab), Libtayo (Cemiplimab-rwlc), Lorbrena (Lorlatinib), Lorlatinib, Mekinist (Trametinib Dimethyl Sulfoxide), Methotrexate Sodium, Mvasi (Bevacizumab), Necitumumab, Nivolumab, Opdivo (Nivolumab), Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Pralsetinib, Ramucirumab, Retevmo (Selpercatinib), Rozlytrek (Entrectinib), Selpercatinib, Tabrecta (Capmatinib Hydrochloride), Tafinlar (Dabrafenib Mesylate), Tagrisso (Osimertinib Mesylate), Tarceva (Erlotinib Hydrochloride), Taxotere (Docetaxel), Tecentriq (Atezolizumab), Tepmetko (Tepotinib Hydrochloride), Tepotinib Hydrochloride, Trametinib Dimethyl Sulfoxide, Trexall (Methotrexate Sodium), Vizimpro (Dacomitinib), Vinorelbine Tartrate, Xalkori (Crizotinib), Yervoy (Ipilimumab), Zirabev (Bevacizumab), Zykadia (Ceritinib), Etopophos (Etoposide Phosphate), Etoposide, Hycamtin (Topotecan Hydrochloride), or Zepzelca (Lurbinectedin), or any combination thereof.

In some cases, a subject agent (one that increases lnc122 activity), is used in a combination therapy (is co-administered) with an immunomodulatory agent. Any convenient immunomodulatory agent can be used. In some cases, the immunomodulatory agent is selected from: an anti-CTLA4 antibody; an anti-PD-1/PD-L1 agent (e.g., an anti-PD-1 antibody, a PD-1-binding reagent such as a PD-L1 or PD-L2 ectodomain, an anti-PD-L1 antibody, a PD-L1-binding reagent such as a PD-1 ectodomain, and the like); a CD40 agonist (e.g., CD40L); a 4-1BB modulator (e.g., a 4-1BB-agonist); an anti-CD47/SIRPA agent (e.g., an anti-CD47 antibody, a CD47-binding reagent such as a SIRPA ectodomain, an anti-SIRPA antibody, a SIRPA-binding reagent such as a CD47 ectodomain, and the like); an inhibitor of TIM3 and/or CEACAM1; an inhibitor of TIM3 and/or CEACAM1; an inhibitor of BTLA and/or CD160; and the like.

iv. Kits

The present disclosure provides kits/systems for carrying out a subject method. Such kits comprise various combinations of components useful in any of the methods described elsewhere herein. In some embodiments a subject kit includes a subject agent that increases lnc122 activity (e.g., a genome targeting fusion protein such as a ZF, a TALE, or a CRISPR effector protein fused to a transcription activator; lnc122; a lnc122 functional equivalent; a nucleic acid encoding lnc122; a nucleic acid encoding an lnc122 functional equivalent; a small molecule; or any combination thereof).

In a further embodiment, the kit comprises the components of an assay for monitoring the effectiveness of a treatment administered to a subject in need thereof, containing instructional material and the components for determining whether the level of Myc in a biological sample obtained from the subject is modulated during or after administration of the treatment.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual with an ongene-negative cancer such as a lung cancer (e.g., lung adenocarcinoma). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

v. Definitions

Unless defined otherwise, all technical and scientific terms used herein have the same meaning as commonly understood by one of ordinary skill in the art to which the invention pertains. In describing and claiming the present invention, the following terminology will be used.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence-in some cases operably linked to a promoter. “Operably linked” refers to a juxtaposition of genetic elements, wherein the elements are in a relationship permitting them to operate in the expected manner. For instance, a promoter is operatively linked to a sequence of interest (the sequence of interest can also be said to be operatively linked to the promoter) if the promoter helps initiate transcription of the sequence of interest. There may be intervening residues between the promoter and sequence of interest so long as this functional relationship is maintained.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Polynucleotides include sequences of deoxyribonucleic acid (DNA) or ribonucleic acid (RNA) or mimetics thereof which may be isolated from natural sources, recombinantly produced or artificially synthesized. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to exhibit non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Instructional material,” as that term is used herein, includes a publication, a recording, a diagram, or any other medium of expression which can be used to communicate the usefulness of the nucleic acid, peptide, polypeptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

By the term “modulating,” as used herein, is meant mediating a detectable increase or decrease in the activity and/or level of a mRNA, polypeptide, or a response in a subject compared with the activity and/or level of the mRNA, polypeptide or response in the subject in the absence of a treatment or compound, and/or compared with the activity and/or level of the mRNA, polypeptide, or response in an otherwise identical but untreated subject. The term encompasses activating, inhibiting and/or otherwise affecting a native signal or response thereby mediating a beneficial therapeutic, prophylactic, or other desired response in a subject, for example, a human. A “nucleic acid” refers to a polynucleotide and includes poly-ribonucleotides and poly-

deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present disclosure contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

Ranges: throughout this disclosure, various aspects may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 2, 1 to 3, 1 to 4, 1 to 5, 2 to 3, 2 to 4, 2 to 5, 2 to 6, etc., as well as individual numbers within that range, for example, 1, 2, 2.7. 3, 4, 5, 5.3, and 6. This applies regardless of the breadth of the range.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “helper virus” for AAV refers to a virus allowing AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used as a helper virus. Numerous adenoviruses of human, non-human mammalian and avian origin are known and available from depositories such as the ATCC. Viruses of the herpes family include, for example, herpes simplex viruses (HSV) and Epstein-Barr viruscs (EBV), as well as cytomegaloviruses (CMV) and pseudorabies viruses (PRV); which are also available from depositories such as ATCC.

“Helper virus function(s)” refers to function(s) encoded in a helper virus genome allowing AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, enter a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g. a replication-competent AAV) refers to an infectious phenotypically wild-type virus, and is replicable in an infected cell (i.e. in the presence of a helper virus or helper virus functions). In the case of AAV, replication competence generally requires the presence of functional AAV packaging genes. In some embodiments, AAV vectors, as described herein, lack of one or more AAV packaging genes and are replication-incompetent in mammalian cells (especially in human cells). In some embodiments, AAV vectors lack any AAV packaging gene sequences, minimizing the possibility of generating replication competent AAV by recombination between AAV packaging genes and an incoming AAV vector. In many embodiments, AAV vector preparations as described herein are those containing few if any replication competent AAV (rcAAV, also referred to as RCA) (e.g., less than about 1 rcAAV per 10² AAV particles, less than about 1 rcAAV per 10⁴ AAV particles, less than about 1 rcAAV per 10⁸ AAV particles, less than about 1 rcAAV per 10¹² AAV particles, or no rcAAV).

“Recombinant,” e.g., as applied to a polynucleotide means, a product of various combinations of cloning, restriction or ligation steps, and other procedures resulting in a molecule distinct and/or different from one found in nature. For example, a recombinant virus can be a viral particle encapsidating a recombinant polynucleotide.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control clements known in the art include, for example, transcriptional regulatory sequences such as promoters and enhancers. A promoter is a DNA region capable under certain conditions of binding RNA polymerase and initiating transcription usually downstream (in the 3′ direction) from the promoter.

“Heterologous” means derived from a genotypically distinct entity from the rest of the entity to it is being compared too. For example, heterologous can be used to refer to a nucleotide or polypeptide sequence that is not found in the native nucleic acid or protein, respectively. For example, when a nucleotide sequence is operably linked to a promoter that it different from the promoter it is naturally operably link to, the promoter can be referred to as a heterologous promoter. In other words, a promoter removed from its native coding sequence and operatively linked to a different coding sequence is heterologous to that sequence. Thus, both the nucleotide sequence and the promoter can be said to be heterologous to one another. As additional examples, a nucleotide sequence introduced by genetic engineering techniques into a plasmid or vector derived from a different species is a heterologous nucleotide sequence (relative to the plasmid or vector). As another example, an exogenous nucleic acid such as a recombinant expression vector introduced into a cell can be said to be heterologous to the cell.

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of the intended functionality. The terms also encompass a modified amino acid polymer; for example, disulfide bond formation, glycosylation, lipidation, phosphorylation, methylation, carboxylation, deamidation, acetylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, retaining the desired biochemical function of the intact protein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell. An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

As used herein, the terms “therapy” or “therapeutic regimen” refer to those activities taken to prevent, treat or alter a disease or disorder, e.g., a course of treatment intended to reduce or eliminate at least one sign or symptom of a disease or disorder using pharmacological, surgical, dietary and/or other techniques. A therapeutic regimen may include a prescribed dosage of one or more compounds and/or or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

A “disease” is a state of health of an animal wherein the animal cannot maintain homeostasis, and wherein if the discasc is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. Subject agents (that increase lnc122 activity) and pharmaceutical compositions thereof can be packaged in single or multiple unit dosage form for ease of administration and uniformity of dosage.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method for treating cancer, comprising:
   administering to an individual who has cancer a composition comprising an agent that increases activity of long non-coding RNA 122 (lnc122) in cancer cells of the individual.
2. The method of 1, wherein said agent comprises a genome targeting fusion protein that causes increased transcription of lnc122 from its endogenous locus.
3. The method of 2, wherein said genome targeting fusion protein is a CRISPRa agent.
4. The method of 2, wherein said genome targeting fusion protein is a Zinc Finger (ZF) or TALE agent.
5. The method of 1, wherein said agent comprises: (i) lnc122, or a functional equivalent thereof; or (ii) a nucleic acid that encodes said lnc122 or encodes said functional equivalent thereof.
6. The method of 5, wherein said functional equivalent thereof comprises a nucleotide sequence having 85% or more sequence identity with the lnc122 sequence set forth as SEQ ID NO: 5 or 6.
7. The method of 5, wherein the agent comprises said lnc122 or said nucleic acid that encodes lnc122.
8. The method of 7, wherein said lnc122 is human lnc122.
9. The method of 8, wherein said lnc122 does not include an intron.
10. The method of 7, wherein said lnc122 is mouse lnc122.
11. The method of 5, wherein said agent comprises a virion that comprises (i) and/or (ii).
12. The method of 5, wherein said nucleic acid is a viral vector.
13. The method of 12, wherein said viral vector is an AAV viral vector.
14. The method of 13, wherein said AAV is AAV8.
15. The method of 5, wherein said nucleic acid is a plasmid DNA, minicircle DNA, or doggybone DNA.
16. The method of 5, wherein the agent comprises a nanoparticle that comprises (i) and/or (ii).
17. The method of 16, wherein the nanoparticle is a lipid nanoparticle (LNP).
18. The method of 1, wherein the cancer is liver cancer, lung cancer, ovarian cancer, prostate cancer, colorectal cancer, renal cancer, bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, gastric cancer, cancer of the central nervous system, skin cancer, head and neck cancer, or blood cancer.
19. The method of 1, wherein the cancer is characterized by Myc deregulation.
20. The method of 19, wherein the cancer is liver cancer, bile duct cancer, bladder cancer, brain cancer, cancer of the nervous system, breast cancer, cervical cancer, colon cancer, esophagus cancer, head and neck cancer, renal cancer, cancer of the large intestine, leukemia, lymphoma, lung cancer, melanoma, skin cancer, ovarian cancer, endometrial cancer, pancreatic cancer, prostate cancer, sarcoma, stomach cancer, testicular cancer, or cancer of the uterus.
21. The method of 1, wherein the cancer is a Myc-driven cancer.
22. The method of 19, wherein the Myc-driven cancer is liver cancer, breast cancer, colorectal cancer, pancreatic cancer, lung cancer, brain cancer, or prostate cancer.
23. The method of 1, wherein the cancer is a liver cancer.
24. The method of 20, wherein the liver cancer is hepatocellular carcinoma (HCC), intrahepatic cholangiocarcinoma, or hepatoblastoma.
25. The method of 1, wherein the cancer is hepatocellular carcinoma (HCC).
26. The method of 1, wherein said administering comprises local administration.
27. The method of 24, wherein said local administration comprises injection into a tumor.
28. The method of 1, wherein said administering comprises systemic administration.
29. The method of 1, wherein said administering comprises intravenous or subcutaneous administration.
30. The method of 1, wherein said method comprises administering a second agent to the individual, wherein the second agent is a cancer targeting agent.
31. The method of 30, wherein the cancer targeting agent specifically binds a cancer cell antigen.
32. The method of 30, wherein the second agent comprises one or more selected from the group consisting of: Atezolizumab, Avastin (Bevacizumab), Bevacizumab, Cabometyx (Cabozantinib-S-Malate), Cabozantinib-S-Malate, Cyramza (Ramucirumab), Infigratinib Phosphate, Keytruda (Pembrolizumab), Lenvatinib Mesylate, Lenvima (Lenvatinib Mesylate), Nexavar (Sorafenib Tosylate), Nivolumab, Opdivo (Nivolumab), Pemazyre (Pemigatinib), Pembrolizumab, Pemigatinib, Ramucirumab, Regorafenib, Sorafenib Tosylate, Stivarga (Regorafenib), Tecentriq (Atezolizumab), and Truseltiq (Infigratinib Phosphate).
33. The method of 1, wherein chemotherapy is administered to the individual.
34. The method of 1, wherein radiotherapy is administered to the individual.
35. A method of reducing proliferation of a target cancer cell, the method comprising:
   introducing into a target cancer cell a composition comprising an agent that increases activity of long non-coding RNA 122 (lnc122) in the target cancer cell,
   wherein said introducing results in reduced proliferation of the target cancer cell.
36. The method of 35, wherein the target cancer cell is in vitro.
37. The method of 35, wherein the target cancer cell is in vivo.
38. The method of 35, wherein said introducing comprises administering said composition to an individual comprising said target cancer cell.
39. The method of 35, wherein said agent comprises a genome targeting fusion protein that causes increased transcription of lnc122 from its endogenous locus.
40. The method of 39, wherein said genome targeting fusion protein is a CRISPRa agent, a Zinc Finger (ZF), or a TALE agent.
41. The method of 35, wherein said agent comprises: (i) lnc122, or a functional equivalent thereof; or (ii) a nucleic acid that encodes said lnc122 or encodes said functional equivalent thereof
42. The method of 41, wherein said functional equivalent thereof comprises a nucleotide sequence having 85% or more sequence identity with the lnc122 sequence set forth as SEQ ID NO: 5 or 6.
43. The method of 41, wherein the agent comprises said lnc122 or said nucleic acid that encodes lnc122.
44. The method of 43, wherein the lnc122 is human lnc122.
45. The method of 41, wherein said agent comprises a virion that comprises (i) and/or (ii).
46. The method of 41, wherein said nucleic acid is a viral vector, a plasmid DNA, a minicircle DNA, or a doggybone DNA.
47. The method of 46, wherein said viral vector is an AAV viral vector.
48. The method of 41, wherein the agent comprises a nanoparticle that comprises (i) and/or (ii).
49. The method of 48, wherein the nanoparticle is a lipid nanoparticle (LNP).
50. The method of 35, wherein the cancer is liver cancer, lung cancer, ovarian cancer, prostate cancer, colorectal cancer, renal cancer, bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, gastric cancer, cancer of the central nervous system, skin cancer, head and neck cancer, or blood cancer.
51. The method of 35, wherein the cancer is characterized by Myc deregulation.
52. The method of 51, wherein the cancer is liver cancer, bile duct cancer, bladder cancer, brain cancer, cancer of the nervous system, breast cancer, cervical cancer, colon cancer, esophagus cancer, head and neck cancer, renal cancer, cancer of the large intestine, leukemia, lymphoma, lung cancer, melanoma, skin cancer, ovarian cancer, endometrial cancer, pancreatic cancer, prostate cancer, sarcoma, stomach cancer, testicular cancer, or cancer of the uterus.
53. The method of 35, wherein the cancer is a Myc-driven cancer.
54. The method of 53, wherein the Myc-driven cancer is liver cancer, breast cancer, colorectal cancer, pancreatic cancer, lung cancer, brain cancer, or prostate cancer.
55. The method of 35, wherein the cancer is a liver cancer.
56. The method of 35, wherein the cancer is hepatocellular carcinoma (HCC).
57. A composition, comprising:
   an agent that increases activity of long non-coding RNA 122 (lnc122) in target cells, wherein said composition is formulated for administration into an individual who has cancer.
58. The composition of 57, wherein said agent comprises a genome targeting fusion protein that causes increased transcription of lnc122 from its endogenous locus.
59. The composition of 58, wherein said genome targeting fusion protein is a CRISPRa, Zinc Finger (ZF), or TALE agent.
60. The composition of 57, wherein said agent comprises: (i) lnc122, or a functional equivalent thereof; or (ii) a nucleic acid that encodes said lnc122 or encodes said functional equivalent thereof.
61. The composition of 60, wherein said functional equivalent thereof comprises a nucleotide sequence having 85% or more sequence identity with the lnc122 sequence set forth as SEQ ID NO: 5 or 6.
62. The composition of 60, wherein the agent comprises said lnc122 or said nucleic acid that encodes lnc122.
63. The composition of 62, wherein the lnc122 is human lnc122.
64. The composition of 60, wherein said agent comprises a virion that comprises (i) and/or (ii).
65. The composition of 60, wherein said nucleic acid is a viral vector, a plasmid DNA, a minicircle DNA, or a doggybone DNA.
66. The composition of 65, wherein said viral vector is an AAV viral vector.
67. The composition of 60, wherein the agent comprises a nanoparticle that comprises (i) and/or (ii).
68. The composition of 67, wherein the nanoparticle is a lipid nanoparticle (LNP)
69. A recombinant viral vector, comprising:
   a nucleotide sequence encoding lnc122 or an equivalent thereof, wherein said nucleotide sequence is operably linked to a heterologous promoter.
70. The recombinant viral vector of 69, wherein the promoter is a constitutive promoter.
71. The recombinant viral vector of 69, wherein the promoter is an inducible promoter
72. The recombinant viral vector of 69, wherein the promoter is a tissue specific promoter
73. The recombinant viral vector of 69, wherein the recombinant viral vector is an AAV vector.
74. The recombinant viral vector of 69, wherein the recombinant viral vector is an AAV8 vector.
75. The recombinant viral vector of 69, wherein the recombinant viral vector is a retroviral vector.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Characterizing lnc122 Function

The independent role of lnc122 as an lncRNA was investigated and the results are reported here. The data suggest the lnc122 is a mediator of UBR5-dependent Myc degradation such that increased lnc122 levels lead to reduced Myc protein levels. When lnc122 levels are decreased, cell proliferation accelerates as does cancer progression. When lnc122 levels are increased, cell proliferation slows and cancer progression is inhibited.

### Results

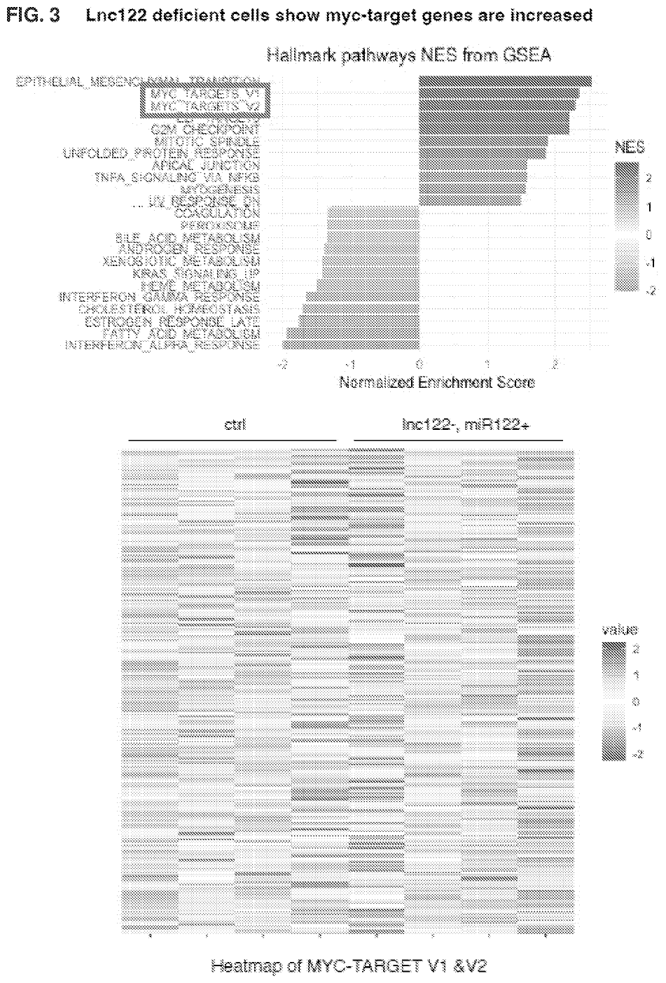

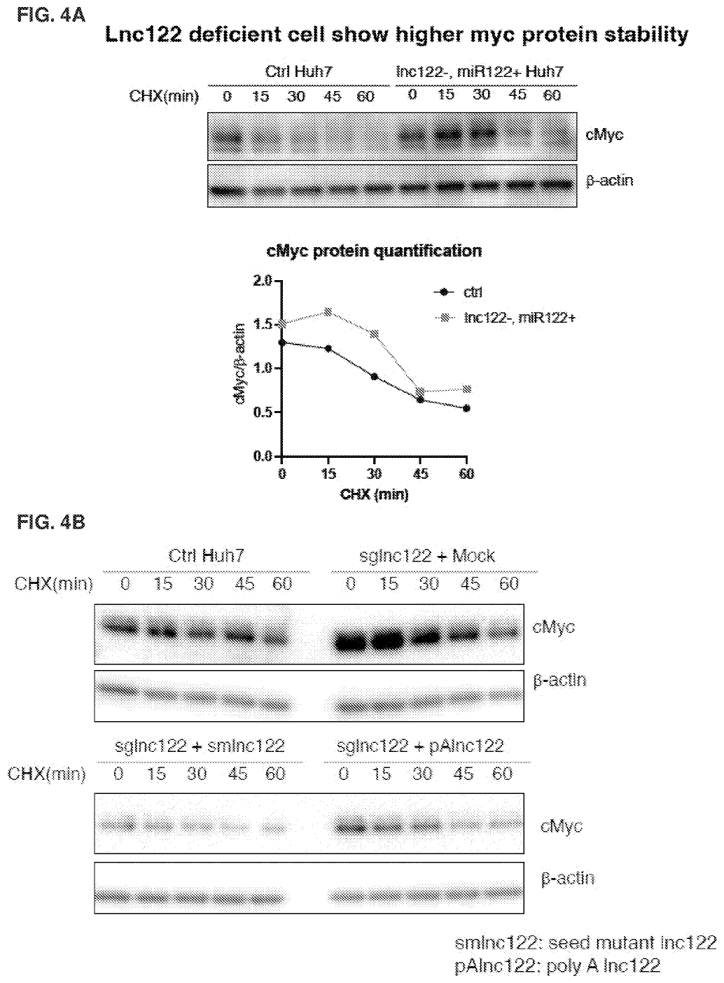

To figure out the specific role of lnc122, an lnc122 specific knockdown cell (lnc122−/miR122+ cell) was gencrated using lenti-crispri_lnc122 and lenti-miR122. First, lnc122−/miR122+ cells were more proliferative (assessed using MTS assay) compared to control cells (FIG. 2). RNAseq of control and lnc122−/miR122+ cells showed that Myc target genes were increased whereas metabolic related genes were reduced (FIG. 3). The RPM (reads per million) of Myc in RNAseq was not changed in lnc122−/miR 122+ cells-and protein stability of Myc in lnc122−/miR122+ cells was also tested. The Myc protein stability was dramatically increased in lnc122−/miR122+ cells indicating lnc122 could regulate the Myc protcin turnover rate (FIG. 4A). Myc protcin stability was decreased when lnc122 expression was increased (FIG. 4B). The sequence of CRISPRi guide RNA guide sequence targeting human lnc122 was 5′-AGAACGGCCTGATCACTCA-3′ (SEQ ID NO:17).

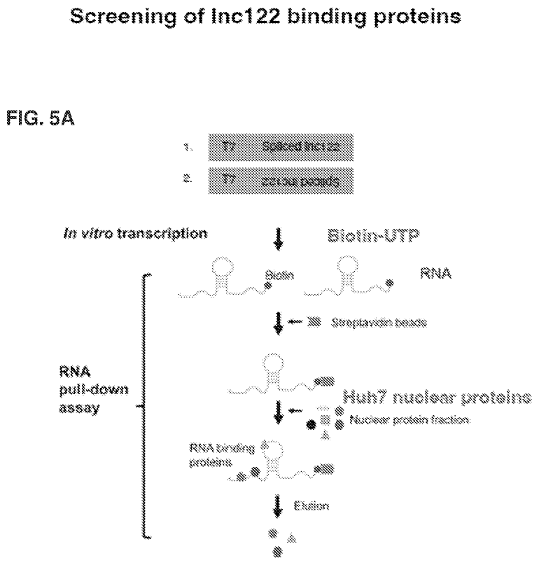

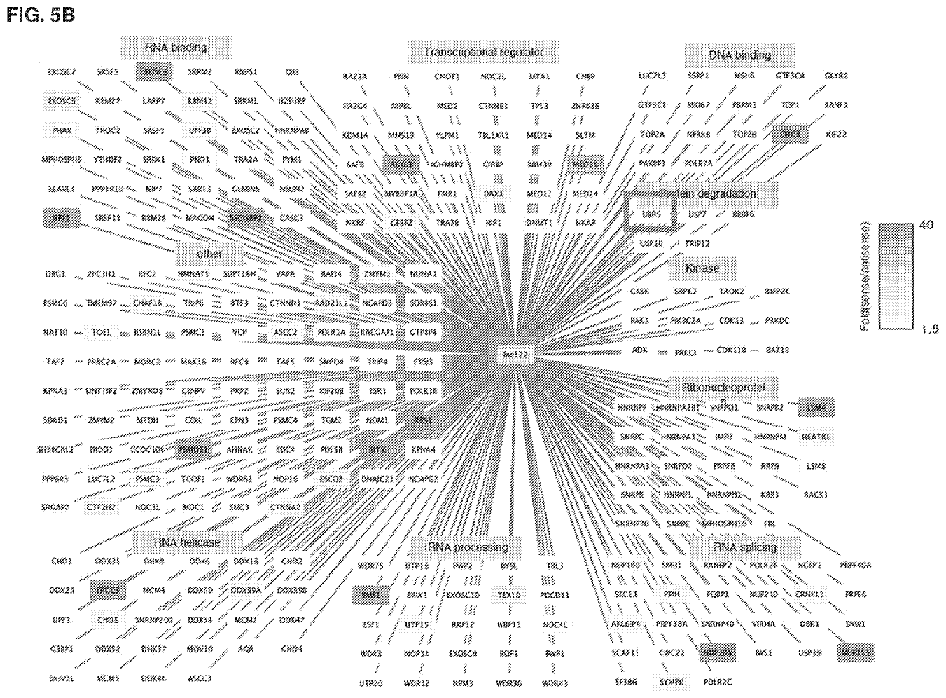

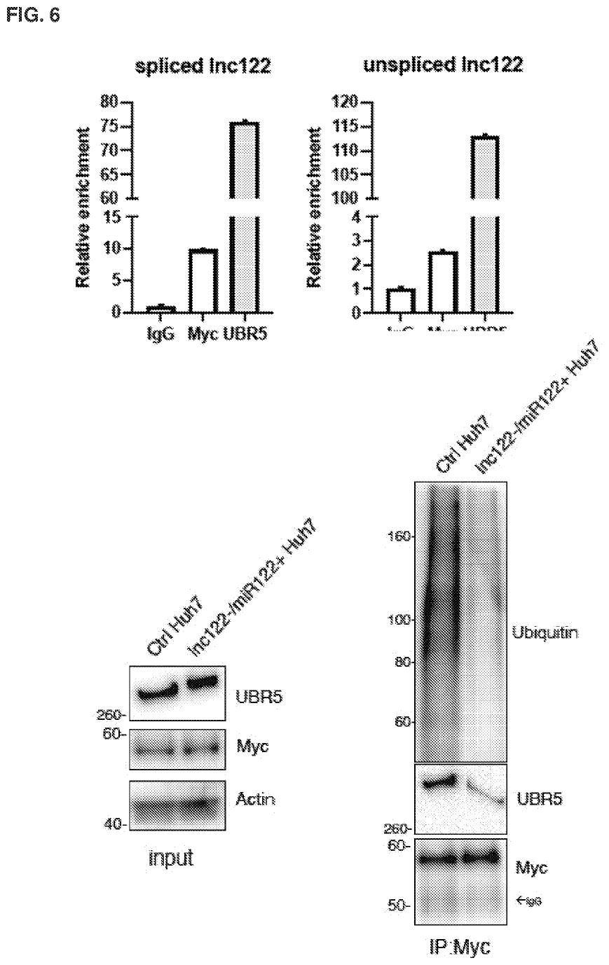

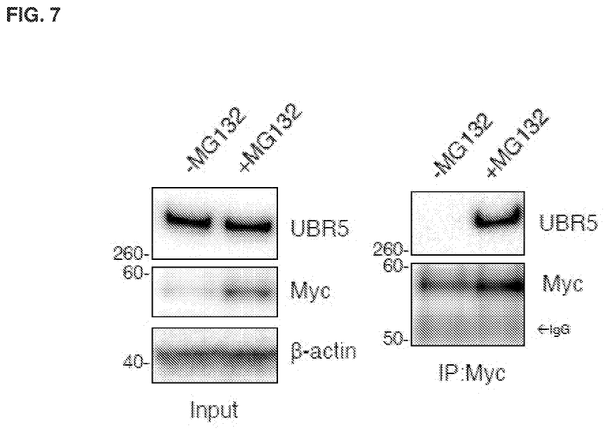

To find out how lnc122 affects the Myc protein stability, the binding proteins of lnc122 were screened using biotinylated RNA pulldown-Mass spectrometry methods (FIG. 5). Among various candidates of lnc122 binding proteins, UBR5, which is reported as a Myc-E3 ubiquitin ligase, was focused on. UBR5 clearly bound with both forms (spliced and unspliced) of lnc122 (FIG. 6), and the interaction of Myc and UBR5 and Myc ubiquitination were significantly reduced in the lnc122−/miR 122+cell indicating lnc122 has a role in Myc protein destabilization (FIG. 6).

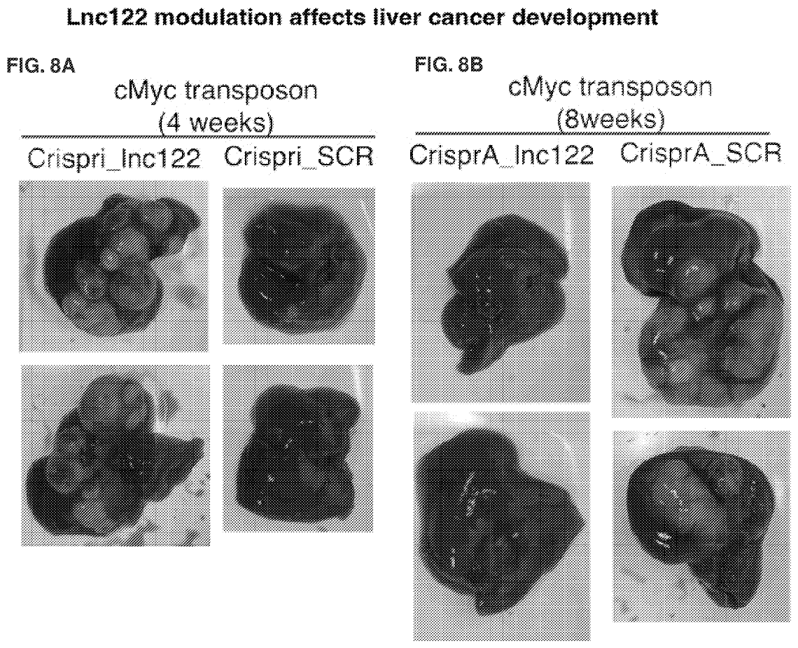

To test the role of lnc122 in tumorigenesis in vivo, a mouse model was generated using sleeping-beauty transposon-mediated Myc overexpression and a CrisprI/CrisprA lnc122 system. Suppression of lnc122 by Crisprl accelerated the liver tumorigenesis whereas induction of lnc122 by CrisprA blocked liver tumor development (FIG. 8). The sequence for CRISPRi guide RNA guide sequence was 5′-AAACCCTGGATCCCATAAAG-3′ (SEQ ID NO:18), and for CRISPRa guide RNA guide sequence was 5′-ACCAAAGGTGACTCTGACTTA-3′ (SEQ ID NO: 19).

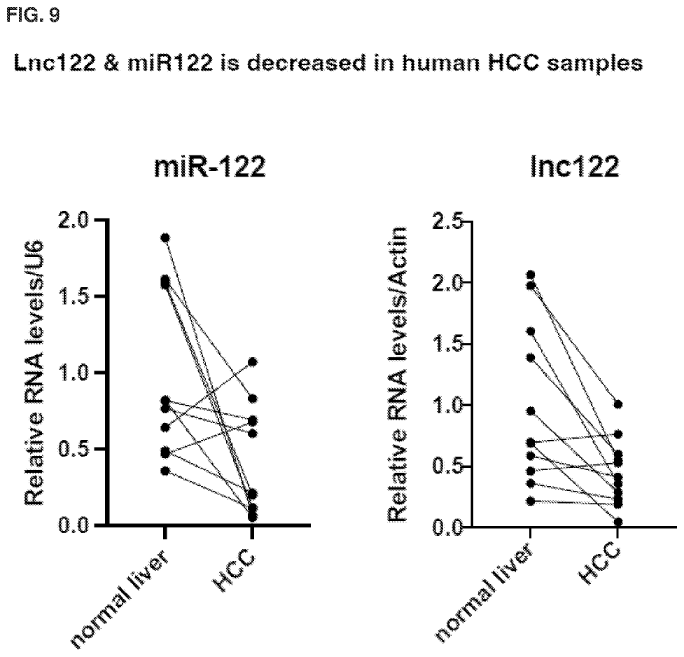

HCC patient samples were obtained from Stanford Tissue Bank and miR122 and lnc122 levels were assessed-both were reduced in HCC compared to its paired normal liver (FIG. 9). In addition, complement of lnc122 in lnc122 deficient cell restored the lower cell proliferation rate and suppressed myc protein levels.

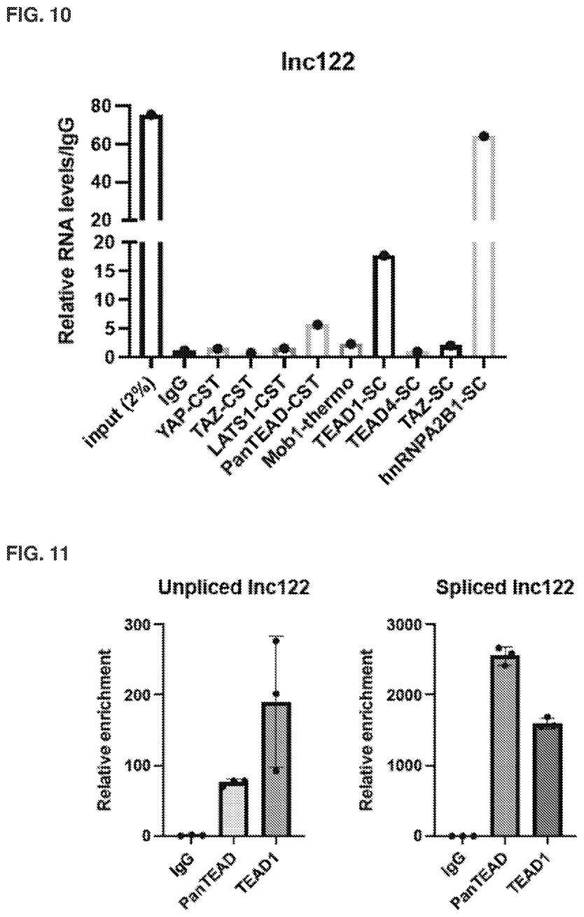

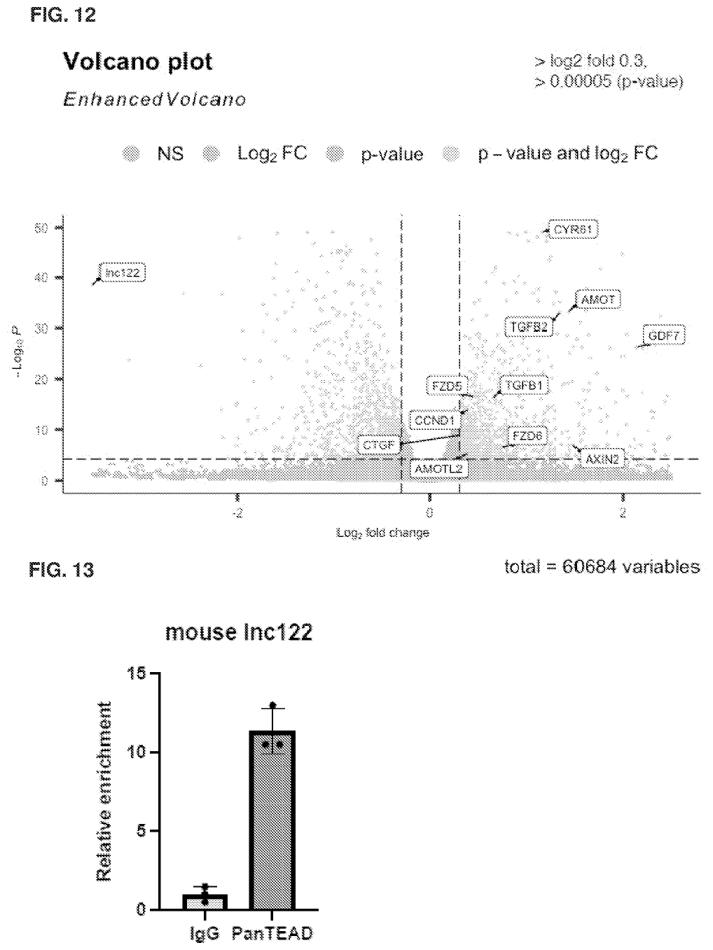

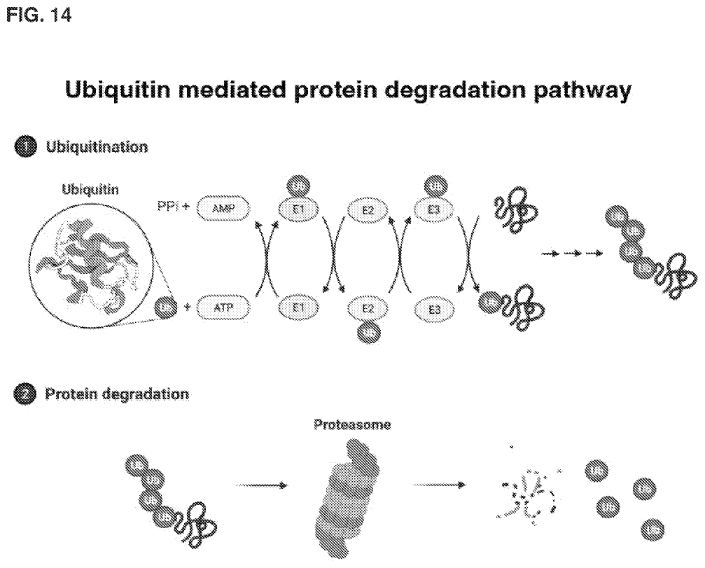

FIGS. 10-13 show that lnc122 also binds with TEAD transcription factor which is important for organ development and in cancer progression. lnc122 deficient cells exhibit increased expression of target genes of TEAD. FIG. 14 is a schematic of the Ubiquitin mediated protein degradation pathway.

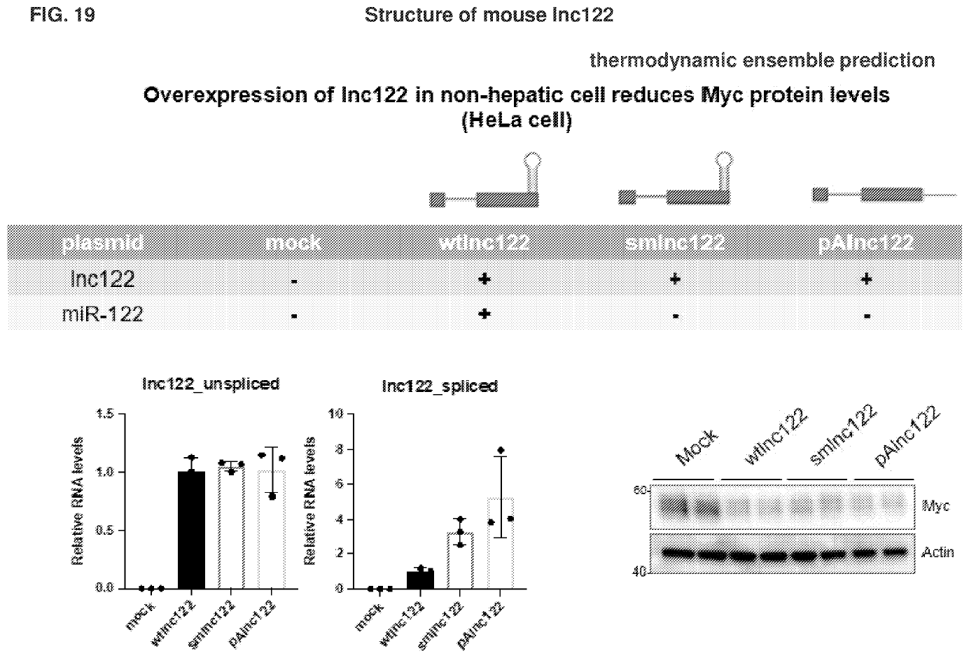

FIG. 19 shows that lnc122 overexpression in non-hepatic cell (HeLa cell) led to reduction in Myc levels. Plasmids which contain mock, wtlnc122(lnc122+/miR122+), smlnc122(lnc122+/miR122−), and pAlnc122(lnc122+/miR122−) were transfected by lipofectamine 3000 reagent (invitrogen). 72 hr after transfection, cells were harvested and Myc protein levels were determined by western blotting.

### Methods

### Generation of Stable Cell Lines and Cell Culture

Huh7 human hepatoma cell lines and HEK293T were cultured in Dulbecco's modified Eagle's medium (DMEM; GIBCO-BRL) with 2 mM L-glutamine, antibiotic/antimycotic and 10% FBS. For generation of modified lnc122 expressing Huh7 cell lines, we performed combinational lentiviral transduction of sgSCR, sglnc122, Mock and miR122 in dSpCas9-KRAB (CRISPRi) expression Huh7 cell. To generate the lentivirus, we cultured the HEK293T cells in 15 cm dish and transfected with 6 ug pMD2. G, 6 ug psPAX2, and 12 ug of dCas9-KRAB (CRISPRi), sgSCR, sglnc122, mock, miR122, smlnc22 and pAlnc122 using polycation polyethylenimine (PEI). After 48 hr, the supernatant was collected and filtered with 0.45 um filter and the virus was concentrated 1:10 using Lenti-X concentrator (Clontech). To generate dCas9-KRAB expressing Huh7 cell, we plated the 2 millions Huh7 cells in 10 cm and adding 200 ul of dCas9-KRAB lentivirus. Two days later, we select the cell using 50 ug/ml of blasticidine for 7 days. To generate modified lnc122 stable cell line, we seeded 300K of dCas9-KRAB expressing Huh7 cell per well of 6 well plate and treated combination of 80 ul concentrated virus (sgRNA, sglnc122) and 40 ul of Mock, miR122, smlnc 122 and pAlnc122. Two days later, we select lentivirus positive cell using 5 ug/ml puromycin and 50 ug/ml hygromycine for 7 days and check the gene expression.

For CRISPRa experiments in mouse, a combination of AAV-dSaCas9-VP64 and AAV-MPH (MS2, p65, HSF1) was used (Sigma Aldrich: CRISPRa Synergistic Activation Mediator (SAM), which uses CRISPR-Cas9-mediated guidance to target SAM components to gene promoters, enabling site-specific transcriptional activation of a gene of interest. Nuclease-dead SaCas9 (dSaCas9), fused to the transcriptional activator VP64, complexes with the CRISPR guide RNA (gRNA). The stem-and tetra-loop sequences in the gRNA scaffold have been modified into minimal hairpin RNA aptamers, which selectively bind dimerized MS2 bacteriophage coat proteins. MS2 coat protein is fused to the p65 subunit of NF-kappaB and the activation domain of human heat-shock factor 1 (HSF1). The guide RNA contains two aptamers, each capable of binding two MS2 coactivator proteins, effectively recruiting four coactivators for every CRISPR targeting activator complex).

### Co-Immunoprecipitation & Ubquitination Assay

Cells were treated with 20uM MG132 (Abcam) for 6 hrs then lysis with IP-lysis buffer (thermo fisher) including 100× of protease inhibitor cocktail (Sigma), and 5 mM N-Ethylmaleimide (NEM) (Calbiochem). Total cell lysates were obtained by centrifugation at 12,000 r.p.m. for 15 min at 4C, and 1-1.5 mg of lysates was used for immunoprecipitation. The lysates were incubated with anti-Myc (Cell signaling technology) for overnight at 4C, followed by 1 h of further incubation with 20 ul protcin G magnetic beads (Invitrogen). After washing three times with the lysis buffer, the immunoprecipitated proteins were recovered from the beads by boiling for 10 min in the sample buffer and analysed by SDS-PAGE and immunoblotting.

RNA Extraction and qRT-PCR

Total RNA was isolated using the TRIzol reagent (Invitrogen) following the manufacturer's protocol. Subsequently, equal amounts of RNA were subjected to cDNA synthesis using superscript IV reverse transcriptase (Thermo Fisher). The relative amount of mRNA was evaluated by using a CFX real-time quantitative PCR detection system (Bio-Rad Laboratories, Hercules, CA, USA) and calculated following normalization to the level of GAPDH or b-actin mRNA.

### Biotinylated RNA Pulldown Assay

Human lnc122 sense and antisense DNA were synthesized with T7 promoter combined primer set. Using PCR products, we synthesized the lnc122 sense RNA and antisense RNA using T7 RNA polymerase (sigma) with biotin-UTP (sigma) and then synthesized RNA were mixed with Huh7 nuclear extracts overnight and pulldown the RNA using magnetic streptavidin beads (Invitrogen). After three times wash, the binding proteins were eluted with SDS sampling buffer and run to 4-12% SDS PAGE gel for 10 min. The gel was stained with Coomassie brilliant blue (Bio-Rad) and then cut and sent the stained band to Mass-spec facility (BGI, San Jose) to analyze binding proteins.

RNA immunoprecipitation

For RNA immunoprecipitation, 3 million cells per replicate were plated in 10 cm dish and performed the nuclear extraction using 500 ul of nuclear extraction buffer (1.28M sucrose, 20 mM Tris-HCL pH 7.5, 20 mM MgC12, 4% Triton X-100). Nuclei were pelleted by centrifugation at 2,500g for 15 min. Nuclear pellet was suspended in IP-lysis buffer (thermo fisher) supplemented with 200× of RiboLock RNase inhibitor (40U/ul), 100× of protease inhibitor cocktail (Sigma), and 5 mM N-Ethylmaleimide (NEM) (Calbiochem). After 15 min max speed spin at 4C, the supernatant was precleared with protein G beads for 30 min at 4C and the bead were discarded. Then the supernatant was incubated with 0.5 ug rabbit IgG (Cell Signaling Technology) or 0.5 ug UBR5 (ab70311, abcam) or Myc (9402S, CST) and rotated overnight at 4C. 10 ul protein G beads were added and rotated for Ihr at 4C and washed three times with IP-lysis buffer (thermo fisher). The protein G beads were subjected with Trizol to extract RNA.

### RNA-Seq

RNA was extracted using Zymo Direct-zol RNA Miniprep Plus Kit with on-column Dnase digestion (Zymo Research). 100ng total RNA was subjected to rRNA depletion kit (NEB) to remove ribosomal RNA and then prepare the RNA-seq library using TruSeq Stranded mRNA Library Prep Kit (Cat #20020594, Illumina) for each sample following the manufacturer's instruction. The library was sequenced on an Illumina partial lane Nova-seq to generate 2X150 paired-end reads

### Measurement of Cell Proliferation

Cell proliferation was measured with a CellTiter 96 nonradioactive cell proliferation assay kit (MTS assay; Promega) according to the manufacturer's instructions. Each independent experiment was performed in 8-12 replicates.

### Western Blotting

Cell lysates were prepared using IP-lysis buffer (Thermo Fisher) or SDS-lysis buffer (1% SDS, 10 mM Tris-HCl pH7.5, 30% glycerol) with protease inhibitor cocktail (Sigma) and 5 mM NEM (Sigma). 10-20 ug of protein lysate was run on a 3-8% or 7% tris-acetate gel and transferred to PVDF membrane using iBlot2 (Thermo Fisher). The membrane was incubated for 20 min at room temperature in a 4% BSA (Omnipur) solution or 5% skim milk and incubated overnight at 4C with indicated antibodies. After washing and incubation for 2h at room temperature with secondary antibody, the protein signal was detected using EMD Millipore Luminata Western HRP substrate (Forte or Crescendo) according to the manufacturer's instructions. Antibodies for immunoblotting were as follows:

### Preparation of Recombinant Adeno-Associated Virus (AAV)

293T cells were transfected using PEI with 3.75 ug AAV8 capsid plasmid, 11.25 ug pAd5 helper plasmid and 3.75 ug of each AAV including dSaCas9-KRAB-U6-sgSCR and dSaCas9-KRAB-U6-sglnc 122 plasmid per 15 cm dishes. AAV pools were purified by double-CsCl gradient centrifugation and calculated the vector genome copy number using qPCR.

### Establishment of Mouse HCC Model

For Crispri mouse experiments, 6 weeks old male FVB/N mice were injected with AAV8-dSaCas9-KRAB-U6-sgSCR (1e12vg/mouse), AAV8-dSaCas9-KRAB-U6-sglnc122 (le12vg/mouse). A week later, Sleeping-Beauty-13 (SB13) plasmid (2 ug/mouse) and T3-cMyc plasmid (20 ug/mouse) were hydrodynamically injected and then 4 weeks later, mice were sacrificed. For CrisprA mouse experiments, 6 weeks old male FVB/N mice were injected with AAV8-dSaCas9-VP64-U6-sgSCR (1e12vg/mouse)+AAV8-MPH (5e11vg/mouse) and AAV8-dSaCas9-KRAB-U6-sglnc122(1e12/mouse)+AAV8-MPH (5e11vg/mouse). A week later, SB13 plasmid (2 ug/mouse) and T3-cMyc plasmid (20 ug/mouse) were hydrodynamically injected and then 8 weeks later, mice were sacrificed.

### REFERENCES

1. P. Ramalingam et al., Biogenesis of intronic miRNAs located in clusters by independent transcription and alternative splicing. RNA 20, 76-87(2014).
2. L. C. Hinske et al., miRIAD-integrating microRNA inter-and intragenic data. Database(Oxford) 2014, (2014).
3. J. L. Rinn, H. Y. Chang, Long Noncoding RNAs: Molecular Modalities to Organismal Functions. Annu Rev Biochem 89, 283-308(2020).
4. Y. Lu et al., lncRNA MIR100HG-derived miR-100 and miR-125b mediate cetuximab resistance via Wnt/beta-catenin signaling. Nat Med 23, 1331-1341(2017).
5. V. Profumo et al., LEADER role of miR-205 host gene as long noncoding RNA in prostate basal cell differentiation. Nat Commun 10, 307(2019).
6. D. He et al., miRNA-independent function of long noncoding pri-miRNA loci. Proc Natl Acad Sci U S A 118, (2021).
7. Q. Sun et al., MIR100 host gene-encoded lncRNAs regulate cell cycle by modulating the interaction between HuR and its target mRNAs. Nucleic Acids Res 46, 10405-10416 (2018).
8. S. Thakral, K. Ghoshal, miR-122 is a unique molecule with great potential in diagnosis, prognosis of liver disease, and therapy both as miRNA mimic and antimir. Curr Gene Ther 15, 142-150(2015).
9. S. H. Hsu et al., Essential metabolic, anti-inflammatory, and anti-tumorigenic functions of miR-122 in liver. J Clin Invest 122, 2871-2883(2012).
10. J. Wen, J. R. Friedman, miR-122 regulates hepatic lipid metabolism and tumor suppression. J Clin Invest 122, 2773-2776(2012).
11. C. Esau et al., miR-122 regulation of lipid metabolism revealed by in vivo antisense targeting. Cell Metab 3, 87-98(2006).
12. A. Dhir, S. Dhir, N. J. Proudfoot, C. L. Jopling, Microprocessor mediates transcriptional termination of long noncoding RNA transcripts hosting microRNAs. Nat Struct Mol Biol 22, 319-327(2015).

### Example 2: Additional Experimental Results

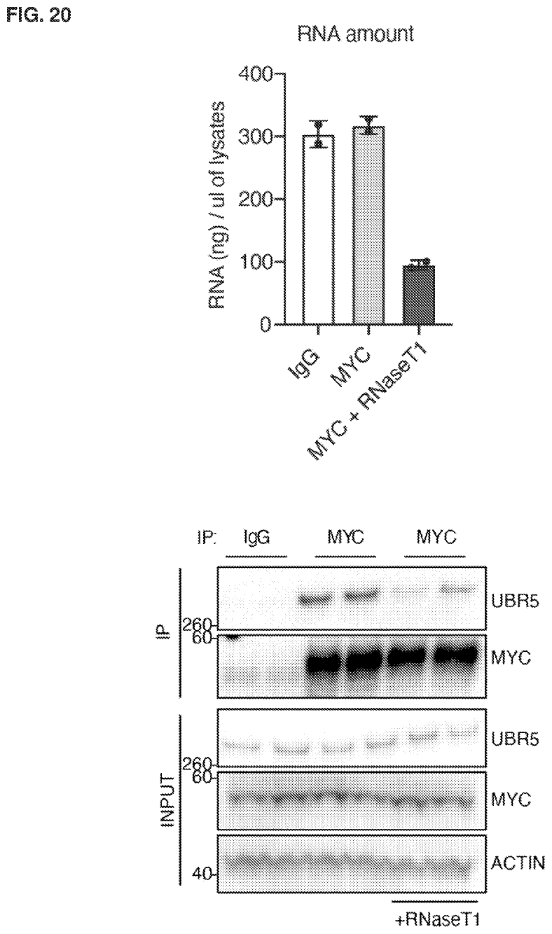

FIG. 20. Huh7 cells were treated with MG132(20 mM) for 4 h. The cell lysates were subjected to immunoprecipitation with IgG, MYC antibody, and MYC antibody with RNaseT1 (25U/ml of lysates) overnight at 4° C. Input and RNA samples were incubated with/without RNaseT1 overnight at 4°° C. This result indicated that there could be some RNAs which are involved in the interaction of MYC and UBR5

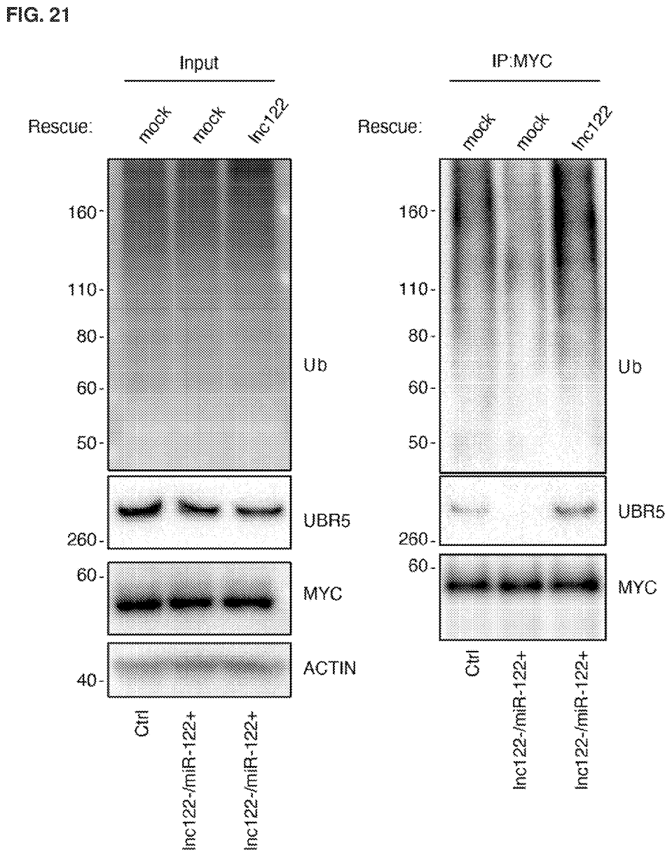

FIG. 21. Control Huh7 cells with Mock plasmid transfection and lnc122−/miR-122+Huh7 cells with Mock and wtlnc 122 plasmid transfection were treated with MG132 (20 mM) for 4 h. The cell lysates were subjected to immunoprecipitation with an anti-MYC antibody overnight at 4° C. IP, immunoprecipitation. This result indicated that in the absence of lnc122, MYC polyubiquitination and binding of MYC and UBR5 are less, but restored by lnc122 complementation.

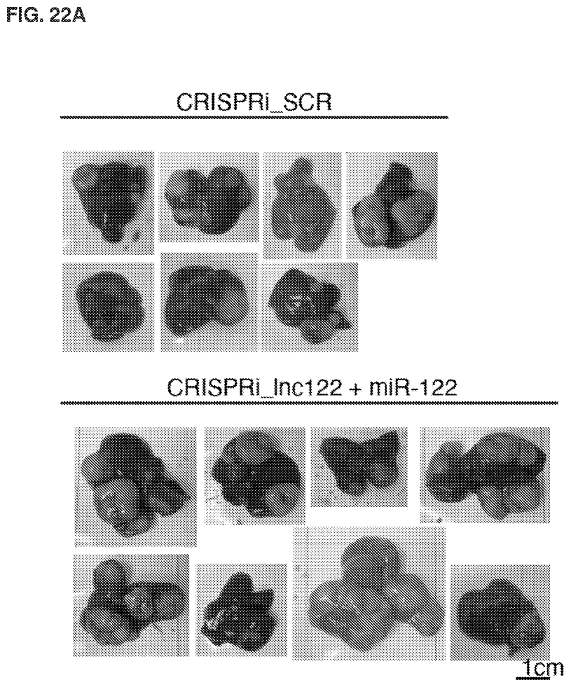

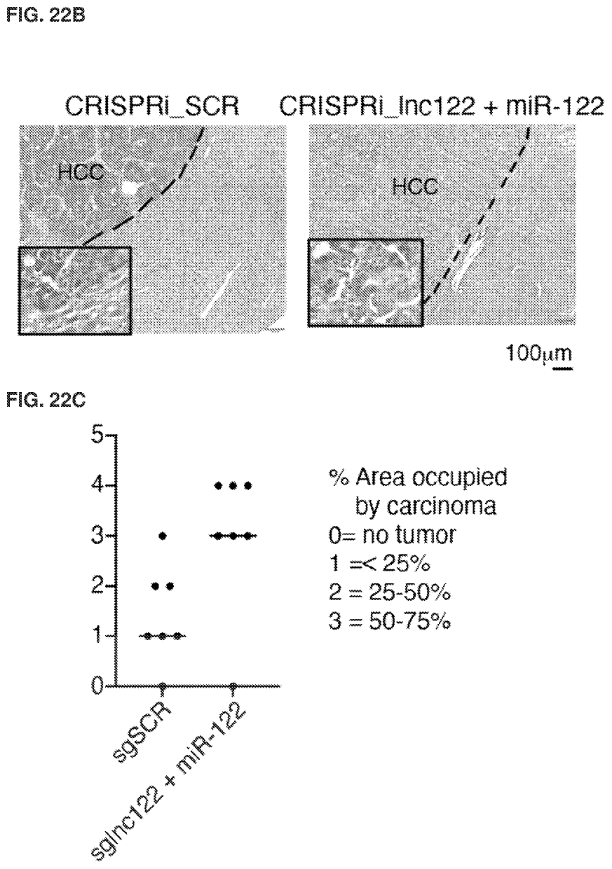

FIGS. 22A-22C. (FIG. 22A) Images of CRISPRi_SCR and CRISPRi_lnc122 +miR-122 injected liver. Scale bar, 1 cm (FIG. 22B) H&E images of CRISPRi_SCR and CRISPRi_lnc122 +miR-122 injected liver. Scale bar, 100 mm (FIG. 22C) H&E staining based hepatic carcinoma area quantification of CRISPRi_SCR and CRISPRi_lnc122 +miR-122 injected liver tumor.

This result indicated that lnc122 has an independent tumor suppressive function regardless of miR-122 in mouse liver.

The disclosures of each and every patent, patent application, and publication cited herein are hereby incorporated herein by reference in their entirety. While this disclosure has been with reference to specific embodiments, it is apparent that other embodiments and variations may be devised by others skilled in the art without departing from the true spirit and scope of the disclosure. The appended claims are intended to be construed to include all such embodiments and equivalent variations. In addition, many modifications may be made to adapt a particular situation, material, composition of matter, process, process step or steps, to the objective, spirit and scope of the present disclosure. All such modifications are intended to be within the scope of the claims appended hereto.

1. A method for treating cancer, comprising:
administering to an individual who has cancer a composition comprising an agent that increases activity of long non-coding RNA 122(lnc122) in cancer cells of the individual.

2. The method of claim 1, wherein said agent comprises a genome targeting fusion protein that causes increased transcription of lnc122 from its endogenous locus.

3. The method of claim 2, wherein said genome targeting fusion protein is a CRISPRa agent, a Zinc Finger (ZF) or TALE agent.

4. (canceled)

5. The method of claim 1, wherein said agent comprises: (i) lnc122, or a functional equivalent thereof; or (ii) a nucleic acid that encodes said lnc122 or encodes said functional equivalent thereof.

6-11. (canceled)

12. The method of claim 5, wherein said nucleic acid is a viral vector, a plasmid DNA, a minicircle DNA, or a doggybone DNA.

13-15. (canceled)

16. The method of claim 5, wherein the agent comprises a nanoparticle that comprises (i) and/or (ii).

17. (canceled)

18. The method of claim 1, wherein the cancer is liver cancer, lung cancer, ovarian cancer, prostate cancer, colorectal cancer, renal cancer, bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, gastric cancer, cancer of the central nervous system, skin cancer, head and neck cancer, or blood cancer.

19. The method of claim 1, wherein the cancer is characterized by Myc deregulation.

20. (canceled)

21. The method of claim 1, wherein the cancer is hepatocellular carcinoma (HCC), intrahepatic cholangiocarcinoma, or hepatoblastoma.

22-27. (canceled)

28. The method of claim 1, wherein said administering comprises systemic administration.

29-34. (canceled)

35. A method of reducing proliferation of a target cancer cell, the method comprising:
introducing into a target cancer cell a composition comprising an agent that increases activity of long non-coding RNA 122(lnc122) in the target cancer cell,
wherein said introducing results in reduced proliferation of the target cancer cell.

36. The method of claim 35, wherein the target cancer cell is in vitro.

37. (canceled)

38. The method of claim 35, wherein said introducing comprises administering said composition to an individual comprising said target cancer cell.

39. The method of claim 35, wherein said agent comprises a genome targeting fusion protein that causes increased transcription of lnc122 from its endogenous locus.

40. The method of claim 39, wherein said genome targeting fusion protein is a CRISPRa agent, a Zinc Finger (ZF), or a TALE agent.

41. The method of claim 35, wherein said agent comprises: (i) lnc122, or a functional equivalent thereof; or (ii) a nucleic acid that encodes said lnc122 or encodes said functional equivalent thereof.

42. The method of claim 41, wherein said functional equivalent thereof comprises a nucleotide sequence having 85% or more sequence identity with the lnc122 sequence set forth as SEQ ID NO: 5 or 6.

43-45. (canceled)

46. The method of claim 41, wherein said nucleic acid is a viral vector, a plasmid DNA, a minicircle DNA, or a doggybone DNA.

47-56. (canceled)

57. A composition, comprising:
an agent that increases activity of long non-coding RNA 122(lnc122) in target cells, wherein said composition is formulated for administration into an individual who has cancer.

58. The composition of claim 57, wherein said agent comprises a genome targeting fusion protein that causes increased transcription of lnc122 from its endogenous locus.

59. (canceled)

60. The composition of claim 57, wherein said agent comprises: (i) lnc122, or a functional equivalent thereof; or (ii) a nucleic acid that encodes said lnc122 or encodes said functional equivalent thereof.

61-68. (canceled)

69. A recombinant viral vector, comprising:
a nucleotide sequence encoding lnc122 or an equivalent thereof, wherein said nucleotide sequence is operably linked to a heterologous promoter.

70-72. (canceled)

73. The recombinant viral vector of claim 69, wherein the recombinant viral vector is an AAV vector or a retroviral vector.

74-75. (canceled)


SEQUENCE LISTING (19 sequences)

SEQ ID NO: 1 - DNA, 4932 bp, Homo sapiens
  source 1..4932: mol_type = unassigned DNA
gaaggcgccatcctgcctgccacaggaagggccttggactgagaggaccggagctgactgggggtaagtgcggctctcccccggcgcctgccgacccccctgagtgatcaggccgttctttggggtggccgctgaccgagaaatgacgggaggctgctgactccggtgttgaaaaagatgccacccagcgcctgagcaacaggaggtgttgaacacaccataactttgtcttccatgattattctgtttcctcagaaagcttgaggaaaactggagactttctttagagagagtaacagatggtttccgtttaaagaaaaataacaaaatggcttaaggaggaaaagaaatgatcaccctgcagagggagagaggatgaggctggggaggagctgagggctcagggacggtgtggggttttatttctgcccttggagtggctggacattaagccagttcttgtttttacgatggcctgattcagcaataacaccaaaccttcaaacaaaatgccggcattagagtcgttcctttggcatgcctctgtccaggtcatattgttccctcaatagcactaaaaatagctggacacctgtgcagaaatgagacagttccttaggaatccccaagcctcagcaattctctttaaaaagagtccgtggaggccccaacttggctaaaaatgtacccagtgattccttcttcaccactgattggtgagaaacacaggcatgaataggtgcatgaatagggaactttccagatttcccttctagatggatttcaatgaggaaagaaatgctgctttgctccctgttgacaagaacattctattttaaaatttccatgcactttcaaaatgacaagactcagaagcataataccaacttcctttgtcatgtagactaacagcctaaaataatcaaatggatgcatttaacacatctattttaaatggcagtaaacgtggattcagagtaaaaagcatctccacttatctttcactttttaagaaatttttttttggtactgatgggttaggtttgcagcaaggtgacacttcacagtccccaaagcacccaaaccccctatggggttggccctagaatgctctcagcccctgggaacaacatcttaaagcaaagacaagctgctcgtgcccagatctgacctgagaaagtgagtgttgcgatattcctggtacttgtgaatctaagacgtcacgctgggagctgtggtgcatacagaatgcgtacagagtggtgaatttgctggagggtctggctttgtaaaagcttcctccttaatgagctttttttctcacttatgaaaagaaatgcagggcattaaaagaggagcaaaccaaatctagcctgtttttcatactcacggatccagtgggaaagctgcattctgaattggccacagcagctagagatgcctggagatttcatcactgttgggaagacgagtgcaggaagatgacagcacacagaaatttcgtgtgtgtgttttctttagaaatgggatctcaaacaactgggctcaagggatcctcctgcctaagactcctgagtagctgagactccaggagaaatttagtattaatagactgccattcaggaatggctagcactctacctttctcagggagatctcaggaagtaaggcataaagagagatggtgggagatttatgtggatgggatcggggcccagggaagctaaacagaatccctcaaggccttttccacactgtagaaaattgtgctggcatttggacaagtcaccctgcttaatatcaaacatctttgccttaagcattattttttcaaaggaaataacgtgttaatacttcccaaccagtgtataatgtttctatttgttattagctcgtactatttaagttcagttgttaagtttgaaaatactagaacattaaaaatctgcagaatgagaaattattttgtgaatgtttaaaatattcaaaagtggatcctgacactaaccacttttaatttattttactagcgatatattattggatgactttttttttttaaaaaaagaaaaccttctgcttgtttgtttaaactgctttgtaaattgacagtttaaccacctctgcagaaacaaagtagtaataaaagatgttggccattttagcaatgtttaaaaattgctcacaagacagaaaatgattggcactcaatatgtgtttagtgattgatgcaataataccataaaattattgtactgcataataaagtatttcactaataatttgctaaaaaattagcctgaggcctgccactcagcacagcactaacttttcatgcttgtgacaagttgtatatgagcaggatgggttccaaccttcccaatttcagaagagtattttgcaagaaggaatttgtattgagagtccagaaagaaaattgcctaaaagtttttaaatcctcatttttctcaataaaaaagtgccatatgaatcataaattaacaaatgcacacaaaaggagaaaatgaaaacaaaccataatttctctactgaaagataaccatggttaacatttgatgttttggcctcaggcatgtatgcattttttcacagaaagggactcatactgtgtatattgttttataaactgttttttaattcaataacatatcttgaatttccaagtctaccagacttttctacagaatcaatttaaataattatggtctatcataaatatattactcttctcctatagctggacatttaggttgtttccaatgtttacccttataaacaatgccactatgagcaaccttataggtaaatctttctacaaatttttgacattttcctggagatagatttctgggccatagaaattgaggttgatttggggtggggggagttggtaagatttacctgtaaaggttgatttggggggtggggagttggtaagatttacctgtaatgcattaacttcctcatctcttttcatcttccatctgttccttcccttttctgtattttgcagattattcaaagccaaaagccaacagagcccccatggagaagtggaggatgctcagatactgctatggagccctgagaagtgaaatgggtccaagcccttgcccaggatagagcgctccaagcgcatcctccggggacccttttaatcagtgaattgttcacctctcactgtttcttcccagcagagctaagagacttctagagcctcttgtggatggtcagcgatagccctggtatgaccaggaggtggcgatttctgttctaggtccctcagcaaagactaagcaggacaagcctggctactgtgttaggaggagctgtagggaaagggaaggacaagatgggccaccacatgcctgtgtccactgctggccacacccagagtgctaggggtttgtggcagtgactggggccacctagagcagttctagggttcgtagtcattctctctgagtcatggaaaactgcccaggaggcgagtcccacctaagagagtatgtgtagcactggcacggcctgctgccttgcaaatgaccacatttcatgaaagaaagccagggccacagacaggaaccaaaggaaatcagtcacttaggggaagataacgaatccacaaagtgaaaaagggaaagaaaaaaagaggcaacttcaacactccaacccatgggagcagaacgactttcagctttcgcagggggataatcactgcaagtccagcttgaagagggtgatttcaggatctacatgagaataaggcgcaattttttttttttttttctgaaaaatacggaagggttcatttacagctgttaaagatgggccggttttagggctaaagcaagactctggtcagatgttaacaactcagttatcctggggaaaaaataactggctcccaaccctttcccttttcagcatttgcaggaaaagaaggagtaggtaaaatgctttatttcctctcattctccatggtgggcaggagttcacataacaattataaaccatctttaacaggaaagccaagtaaatcctgtagggatttatttggagctccctttttgctagaaaatgagtatcatgaactaaaaatggactcgtttcctctctctccatcttcctctctttcttctctcccctcatctcccacccaccgccatggtggaaaatgactgtggtcaagaagattgaacatttgagcaagaccagatgcctttatatccaatagatgccacggagagtagatcacaacacatctaactgctgtatccccatataaaataattttaattttcctagactgcagaattgatcacatttactgatgatggattgggaagtaggtgaagaggtgagagttggagagtatccattcattcggccagattattctcatttataatgtagtcttccaataagtatttatttccagaaatctattccctctagagattctttctctctcagccattttagaaaattgttgcaaacagagttcctgtccattttcacaggaagtttttgcagaaggggtttgctgggtggcagtggaacacaggcagtggacccctctactactctactgttccagaacacagaagcctttcattgagagtgttcagctcttcccattgctcaagatgcttgtacccgtgatgcttcttttctctgcttaggtcacaatatgtggagctgacaaggttcccctattatcagtgacaatggtggaatgtggaggtgaagttaacaccttcgtggctacagagtttccttagcagagctgtggagtgtgacaatggtgtttgtgtctaaactatcaaacgccattatcacactaaatagctactgctaggcaatccttccctcgataaatgtcttggcatcgtttgctttgagcaagaa

SEQ ID NO: 2 - DNA, 1963 bp, Homo sapiens
  source 1..1963: mol_type = unassigned DNA
gaaggcgccatcctgcctgccacaggaagggccttggactgagaggaccggagctgactggggggattattcaaagccaaaagccaacagagcccccatggagaagtggaggatgctcagatactgctatggagccctgagaagtgaaatgggtccaagcccttgcccaggatagagcgctccaagcgcatcctccggggacccttttaatcagtgaattgttcacctctcactgtttcttcccagcagagctaagagacttctagagcctcttgtggatggtcagcgatagccctggtatgaccaggaggtggcgatttctgttctaggtccctcagcaaagactaagcaggacaagcctggctactgtgttaggaggagctgtagggaaagggaaggacaagatgggccaccacatgcctgtgtccactgctggccacacccagagtgctaggggtttgtggcagtgactggggccacctagagcagttctagggttcgtagtcattctctctgagtcatggaaaactgcccaggaggcgagtcccacctaagagagtatgtgtagcactggcacggcctgctgccttgcaaatgaccacatttcatgaaagaaagccagggccacagacaggaaccaaaggaaatcagtcacttaggggaagataacgaatccacaaagtgaaaaagggaaagaaaaaaagaggcaacttcaacactccaacccatgggagcagaacgactttcagctttcgcagggggataatcactgcaagtccagcttgaagagggtgatttcaggatctacatgagaataaggcgcaattttttttttttttttctgaaaaatacggaagggttcatttacagctgttaaagatgggccggttttagggctaaagcaagactctggtcagatgttaacaactcagttatcctggggaaaaaataactggctcccaaccctttcccttttcagcatttgcaggaaaagaaggagtaggtaaaatgctttatttcctctcattctccatggtgggcaggagttcacataacaattataaaccatctttaacaggaaagccaagtaaatcctgtagggatttatttggagctccctttttgctagaaaatgagtatcatgaactaaaaatggactcgtttcctctctctccatcttcctctctttcttctctcccctcatctcccacccaccgccatggtggaaaatgactgtggtcaagaagattgaacatttgagcaagaccagatgcctttatatccaatagatgccacggagagtagatcacaacacatctaactgctgtatccccatataaaataattttaattttcctagactgcagaattgatcacatttactgatgatggattgggaagtaggtgaagaggtgagagttggagagtatccattcattcggccagattattctcatttataatgtagtcttccaataagtatttatttccagaaatctattccctctagagattctttctctctcagccattttagaaaattgttgcaaacagagttcctgtccattttcacaggaagtttttgcagaaggggtttgctgggtggcagtggaacacaggcagtggacccctctactactctactgttccagaacacagaagcctttcattgagagtgttcagctcttcccattgctcaagatgcttgtacccgtgatgcttcttttctctgcttaggtcacaatatgtggagctgacaaggttcccctattatcagtgacaatggtggaatgtggaggtgaagttaacaccttcgtggctacagagtttccttagcagagctgtggagtgtgacaatggtgtttgtgtctaaactatcaaacgccattatcacactaaatagctactgctaggcaatccttccctcgataaatgtcttggcatcgtttgctttgagcaagaa

SEQ ID NO: 3 - DNA, 4980 bp, Mus musculus
  source 1..4980: mol_type = unassigned DNA
cctcccagtccacctgcagacggcgtataccacgtagcagagggggcatggggggagctggaccttcggttttgcttagtgtttgaccaaaggtgactctgacttagtggactaaggtcatgccctctcttcccccacaatcgataatttaatgtgacttatcagaaagagaacgaattgtttacttttaaaccctggatcccataaagggagagtgcacaaaggcctaaaagccacagatgctcggtgctctcacgcacgcaaggctgccctcaaaccctcagtagagagaagataaacactgtcggaggtaagtgccttcttccagggcctacagtgggagtgaggctgcctgccgaggggagagtgagttgcgggctcaggtgtttaaaagccgccacacagctcctgagcaacactgggcataaaacattccctaattgaatttggtctgccatgattattcattctgttcagaaagcctgagcaaaactggacactttctgaaagcttctttatactccctagagaggggaaggggaaggggaaggctagagacatatggaagacgcagaggctggcttgtatttctatccttggagtggctatggtaagccatgtctcatttaagaatggcctggttatcaatagcatcagggccttggatgaagattctacctgtctgtttaaaatgggggtttgtttgacaggggctcctctttgtgtgtgcttcaccatgccttattcttatttcaaagacacctgatcccaattcttcctcgtgtgtgttccaacttgtactgtcccctcaatgatgctagaaatagctgtccacctgcatagaggtgaaactggtccctaggagtcccgagcttcggcagcccttgattgagagtccatcgagcctttaatgctgagaacctggcggaggattctctctttgcctctggttggtgactaaatggccgatttgattgatgtatggataaggaacttttccagatttctccctttctaaatgggtttccttgaaaagaaaagtgactctgttccctggtgaaaacaagaacattctattttaaaagttctgtgtgctttcaaagatgacaagactcgggagatatgaactgactttctttgtctagactgtactagcctaaaacagccaaatggatacatttcacacaacaacttcacatgacagtaaatgcaggtttgcaatttttttttaaagcatctctgctttttgctttttaagaaatccttttgtggtattgatgggttattttttgcagcaaagtgactccacatagttcccgaagctcccccttcttggtcctgggaagctgtttgcaggatgttcttcgctcctttgaacacgctgaaaatacagattcgtgatcacctggtacttgtgaatccaccacatcttcctaggcgctgtggcgcgcgctgatgcatctgaggcaggttacgctttgccaaggcctcctccttagagaccgcccactcccacttaggaagagagatagatgcctggcattagacagggagcaaacggagtttaacttgtttttcatgcacgggggctcaatggggaaactccatttggaattgtccacagcccctagaaatgtctagaaatcccatcctcttggagaaggggaggacaggacacagaaatcttgttttaataggcagacactaagggacccttgtcactcagcctcccttagggaggacttaggaaataaggcactctgactgctaaatggtaggaggtgtgtgggtctgagctcaagagcatgaaccccttcctgcgctacggaaaccctgctggcatttggacacatcaccctgattaacatccaacaccattggtcttagtcattgctttgttgatgttgtttgttttttttgggagggtgggggtcgggggatgttcatagaaaataacagatgctaccatttttccatccagcatgtgcactttctgttggatattagctagtacaagtgagcttcccagtgttcagtctgaaaattctaaagcccttttaagtctgaagaaaggaaacagacaatgtggcttggaccctagaacgaatggctttggcttttcttctcggtgatacattcattagtaaacaacatttggaagaacaggaaaatattctgctgacagaatttgatcacttggtcaaagaaaacacagaaaaacaaaacaaaacaaatgtttcagtagtgtctaaaaattgttcaaagggccctaataaatgcgaatttatttcttgatataatagcataaagttaactatgctgcatagtacttcagtaattatttgcttaaaaaaaattttttttgacctgaagtctctctctgtcagcaagactgtttcatggtggcaaatacatctgtttgagtcagataggttccagctttcatatttacgaaaagtgtttacaaggaggatttattaaatggggttatccagaaggaggagggaggactttgaaatttttaaaattctcattatttttctcatttccaaagcactgtgaaaaaaattgtgaaaattaataaattcaatgaaacagaggtaaaagtccaacagcagctccctttccacctggtcgagcgctacagtaacatgaacgctgtacagttctccctgaaaagggtccgaatcatatgcactgttttgaaaaccactcctctgttggaaatgaaatcttactttgtttccaagtcaacaacagccaactttttaccgagtcgatgattgcaaggtgaggaccgtggtccaggtgagtgtcgaccatttcccaaagcatgagccatgtgcaaccctttattttaaataatgtcactatgaattagctcttatttactcatttagttagttagttagttagtttaccttttccttgagatgtgtgcttgattcatgacctgcccgaaaggcagagaggttaggggagggttgcgtgtaatatgttggttggttccctcctgttcttcctcctctgtcttttgcacatcaagcactgagaggtcctcagcccctacagcactaatgaatgcctcaccgctcctggtcggccttactacacgtgggaagggggttctagagacctgctgagatggtcagagcatccccgggagggggtgagggatgaaaaagaggtgggatttcagtcctaggtcctccaggcagagaaataaatcagcatagctggtctcttggaaggagtggtggggaaagacaggtcatcacctacacacacacacacacacacacacacacacacacacacacacacacacacacactctcgaggtcccccatcattgcatggccagcacaaaggttttagggtggcgctgggtgtcagcaggagcttcctggtttcacagaggagtgcttgtctaacactaggctgagtgacactcttttcctgcagtctgctggggccacagaggaacaaaggaagccaactattatattttattttttaattttttttagtggacaacaatcctgtcagcaccatagaaaaggaaaggggcgaagaggagccaaagtgggagggaggtcggaggtcacagaatgctgttcctgcggcatctgtcttaaaagggtgtctaaaaggtgtcttaaggtggtctctatgggaatacaggatagaagaatgcgggaggactcgtttcgcttgttacagtttaagcaaatctctagccaaatgttaacaactcagctattctggggatgggggtgagtcaggaaactggcacccaatcttcccccttctcaacatttggggaaaataaaggattaggtaaaactcttttcgttcctctcattctccaccgtgagcagaggttcacagaaccggtgaaaccgtatttaactggagacaagtcaatccagaaggcatttgctcacacagcaaccttcccactagaaagtgaaccttcccgctagaaaaaaaaagtgctaactgtttttatttcattctttcttagttggacccctcacctgctgcctgcagttcttctaaagaatggctgtgcagaatacgacttaaagtttgagcaaagtggggtgtctctaaattcaaagattccacagagaggaggtcctagcccataaagaataatcacaagctgggagtagggggtgtgtgcctttcatcctaatacccaagaggcaggcataggtggatctctgtgattttgagtccagttgggtcgatatagtgagttcaaggccagccagggctacatactaagagaccattgtctcaaaagatagaaaaagaaaagaaaagaagagaagagaaaaggaaaggaagggaagggaaggaaaaggaaaggaaaggaaaggaaaggaaaggaaaggaaaggaaaggaaaggaaaggaaaggaaaggaaaggaaaggaagtcataattttcttgaaccaaagaagtgatcacatttgctagtgatggattggaaaccatgacgaggtgagagtgggtgggatctatcacctaatggagttatttccttgtatgatgtggtttgtaagaagtgtctgcctcttcctggaattcaagccttttaggaaacagatgtaaacaaatttccatcctggtaaaggcctttgtgagccctccttgttattcaagatcccggggctcttccctatgctccagtttcagcgtttggaaccactgaggagtctgtgacagacaacggaggaatgtgaagggaaggttaatacccttgtgcctgacagactttccttagcagagctgtggagtgtgacaatggtgtttgtgtccaaaccatcaaacgccattatcacactaaatagctactgctaggcaatccgtccactccacgcgtgacttgacgtctgttctctccgagcaagaagttctgtcttgtgctagagttctccattgtgtgtaatgtatgaataaaatctggtcc

SEQ ID NO: 4 - RNA, 4932 nt, Homo sapiens
  source 1..4932: mol_type = unassigned RNA
gaaggcgccatcctgcctgccacaggaagggccttggactgagaggaccggagctgactgggggtaagtgcggctctcccccggcgcctgccgacccccctgagtgatcaggccgttctttggggtggccgctgaccgagaaatgacgggaggctgctgactccggtgttgaaaaagatgccacccagcgcctgagcaacaggaggtgttgaacacaccataactttgtcttccatgattattctgtttcctcagaaagcttgaggaaaactggagactttctttagagagagtaacagatggtttccgtttaaagaaaaataacaaaatggcttaaggaggaaaagaaatgatcaccctgcagagggagagaggatgaggctggggaggagctgagggctcagggacggtgtggggttttatttctgcccttggagtggctggacattaagccagttcttgtttttacgatggcctgattcagcaataacaccaaaccttcaaacaaaatgccggcattagagtcgttcctttggcatgcctctgtccaggtcatattgttccctcaatagcactaaaaatagctggacacctgtgcagaaatgagacagttccttaggaatccccaagcctcagcaattctctttaaaaagagtccgtggaggccccaacttggctaaaaatgtacccagtgattccttcttcaccactgattggtgagaaacacaggcatgaataggtgcatgaatagggaactttccagatttcccttctagatggatttcaatgaggaaagaaatgctgctttgctccctgttgacaagaacattctattttaaaatttccatgcactttcaaaatgacaagactcagaagcataataccaacttcctttgtcatgtagactaacagcctaaaataatcaaatggatgcatttaacacatctattttaaatggcagtaaacgtggattcagagtaaaaagcatctccacttatctttcactttttaagaaatttttttttggtactgatgggttaggtttgcagcaaggtgacacttcacagtccccaaagcacccaaaccccctatggggttggccctagaatgctctcagcccctgggaacaacatcttaaagcaaagacaagctgctcgtgcccagatctgacctgagaaagtgagtgttgcgatattcctggtacttgtgaatctaagacgtcacgctgggagctgtggtgcatacagaatgcgtacagagtggtgaatttgctggagggtctggctttgtaaaagcttcctccttaatgagctttttttctcacttatgaaaagaaatgcagggcattaaaagaggagcaaaccaaatctagcctgtttttcatactcacggatccagtgggaaagctgcattctgaattggccacagcagctagagatgcctggagatttcatcactgttgggaagacgagtgcaggaagatgacagcacacagaaatttcgtgtgtgtgttttctttagaaatgggatctcaaacaactgggctcaagggatcctcctgcctaagactcctgagtagctgagactccaggagaaatttagtattaatagactgccattcaggaatggctagcactctacctttctcagggagatctcaggaagtaaggcataaagagagatggtgggagatttatgtggatgggatcggggcccagggaagctaaacagaatccctcaaggccttttccacactgtagaaaattgtgctggcatttggacaagtcaccctgcttaatatcaaacatctttgccttaagcattattttttcaaaggaaataacgtgttaatacttcccaaccagtgtataatgtttctatttgttattagctcgtactatttaagttcagttgttaagtttgaaaatactagaacattaaaaatctgcagaatgagaaattattttgtgaatgtttaaaatattcaaaagtggatcctgacactaaccacttttaatttattttactagcgatatattattggatgactttttttttttaaaaaaagaaaaccttctgcttgtttgtttaaactgctttgtaaattgacagtttaaccacctctgcagaaacaaagtagtaataaaagatgttggccattttagcaatgtttaaaaattgctcacaagacagaaaatgattggcactcaatatgtgtttagtgattgatgcaataataccataaaattattgtactgcataataaagtatttcactaataatttgctaaaaaattagcctgaggcctgccactcagcacagcactaacttttcatgcttgtgacaagttgtatatgagcaggatgggttccaaccttcccaatttcagaagagtattttgcaagaaggaatttgtattgagagtccagaaagaaaattgcctaaaagtttttaaatcctcatttttctcaataaaaaagtgccatatgaatcataaattaacaaatgcacacaaaaggagaaaatgaaaacaaaccataatttctctactgaaagataaccatggttaacatttgatgttttggcctcaggcatgtatgcattttttcacagaaagggactcatactgtgtatattgttttataaactgttttttaattcaataacatatcttgaatttccaagtctaccagacttttctacagaatcaatttaaataattatggtctatcataaatatattactcttctcctatagctggacatttaggttgtttccaatgtttacccttataaacaatgccactatgagcaaccttataggtaaatctttctacaaatttttgacattttcctggagatagatttctgggccatagaaattgaggttgatttggggtggggggagttggtaagatttacctgtaaaggttgatttggggggtggggagttggtaagatttacctgtaatgcattaacttcctcatctcttttcatcttccatctgttccttcccttttctgtattttgcagattattcaaagccaaaagccaacagagcccccatggagaagtggaggatgctcagatactgctatggagccctgagaagtgaaatgggtccaagcccttgcccaggatagagcgctccaagcgcatcctccggggacccttttaatcagtgaattgttcacctctcactgtttcttcccagcagagctaagagacttctagagcctcttgtggatggtcagcgatagccctggtatgaccaggaggtggcgatttctgttctaggtccctcagcaaagactaagcaggacaagcctggctactgtgttaggaggagctgtagggaaagggaaggacaagatgggccaccacatgcctgtgtccactgctggccacacccagagtgctaggggtttgtggcagtgactggggccacctagagcagttctagggttcgtagtcattctctctgagtcatggaaaactgcccaggaggcgagtcccacctaagagagtatgtgtagcactggcacggcctgctgccttgcaaatgaccacatttcatgaaagaaagccagggccacagacaggaaccaaaggaaatcagtcacttaggggaagataacgaatccacaaagtgaaaaagggaaagaaaaaaagaggcaacttcaacactccaacccatgggagcagaacgactttcagctttcgcagggggataatcactgcaagtccagcttgaagagggtgatttcaggatctacatgagaataaggcgcaattttttttttttttttctgaaaaatacggaagggttcatttacagctgttaaagatgggccggttttagggctaaagcaagactctggtcagatgttaacaactcagttatcctggggaaaaaataactggctcccaaccctttcccttttcagcatttgcaggaaaagaaggagtaggtaaaatgctttatttcctctcattctccatggtgggcaggagttcacataacaattataaaccatctttaacaggaaagccaagtaaatcctgtagggatttatttggagctccctttttgctagaaaatgagtatcatgaactaaaaatggactcgtttcctctctctccatcttcctctctttcttctctcccctcatctcccacccaccgccatggtggaaaatgactgtggtcaagaagattgaacatttgagcaagaccagatgcctttatatccaatagatgccacggagagtagatcacaacacatctaactgctgtatccccatataaaataattttaattttcctagactgcagaattgatcacatttactgatgatggattgggaagtaggtgaagaggtgagagttggagagtatccattcattcggccagattattctcatttataatgtagtcttccaataagtatttatttccagaaatctattccctctagagattctttctctctcagccattttagaaaattgttgcaaacagagttcctgtccattttcacaggaagtttttgcagaaggggtttgctgggtggcagtggaacacaggcagtggacccctctactactctactgttccagaacacagaagcctttcattgagagtgttcagctcttcccattgctcaagatgcttgtacccgtgatgcttcttttctctgcttaggtcacaatatgtggagctgacaaggttcccctattatcagtgacaatggtggaatgtggaggtgaagttaacaccttcgtggctacagagtttccttagcagagctgtggagtgtgacaatggtgtttgtgtctaaactatcaaacgccattatcacactaaatagctactgctaggcaatccttccctcgataaatgtcttggcatcgtttgctttgagcaagaa

SEQ ID NO: 5 - RNA, 1963 nt, Homo sapiens
  source 1..1963: mol_type = unassigned RNA
gaaggcgccatcctgcctgccacaggaagggccttggactgagaggaccggagctgactggggggattattcaaagccaaaagccaacagagcccccatggagaagtggaggatgctcagatactgctatggagccctgagaagtgaaatgggtccaagcccttgcccaggatagagcgctccaagcgcatcctccggggacccttttaatcagtgaattgttcacctctcactgtttcttcccagcagagctaagagacttctagagcctcttgtggatggtcagcgatagccctggtatgaccaggaggtggcgatttctgttctaggtccctcagcaaagactaagcaggacaagcctggctactgtgttaggaggagctgtagggaaagggaaggacaagatgggccaccacatgcctgtgtccactgctggccacacccagagtgctaggggtttgtggcagtgactggggccacctagagcagttctagggttcgtagtcattctctctgagtcatggaaaactgcccaggaggcgagtcccacctaagagagtatgtgtagcactggcacggcctgctgccttgcaaatgaccacatttcatgaaagaaagccagggccacagacaggaaccaaaggaaatcagtcacttaggggaagataacgaatccacaaagtgaaaaagggaaagaaaaaaagaggcaacttcaacactccaacccatgggagcagaacgactttcagctttcgcagggggataatcactgcaagtccagcttgaagagggtgatttcaggatctacatgagaataaggcgcaattttttttttttttttctgaaaaatacggaagggttcatttacagctgttaaagatgggccggttttagggctaaagcaagactctggtcagatgttaacaactcagttatcctggggaaaaaataactggctcccaaccctttcccttttcagcatttgcaggaaaagaaggagtaggtaaaatgctttatttcctctcattctccatggtgggcaggagttcacataacaattataaaccatctttaacaggaaagccaagtaaatcctgtagggatttatttggagctccctttttgctagaaaatgagtatcatgaactaaaaatggactcgtttcctctctctccatcttcctctctttcttctctcccctcatctcccacccaccgccatggtggaaaatgactgtggtcaagaagattgaacatttgagcaagaccagatgcctttatatccaatagatgccacggagagtagatcacaacacatctaactgctgtatccccatataaaataattttaattttcctagactgcagaattgatcacatttactgatgatggattgggaagtaggtgaagaggtgagagttggagagtatccattcattcggccagattattctcatttataatgtagtcttccaataagtatttatttccagaaatctattccctctagagattctttctctctcagccattttagaaaattgttgcaaacagagttcctgtccattttcacaggaagtttttgcagaaggggtttgctgggtggcagtggaacacaggcagtggacccctctactactctactgttccagaacacagaagcctttcattgagagtgttcagctcttcccattgctcaagatgcttgtacccgtgatgcttcttttctctgcttaggtcacaatatgtggagctgacaaggttcccctattatcagtgacaatggtggaatgtggaggtgaagttaacaccttcgtggctacagagtttccttagcagagctgtggagtgtgacaatggtgtttgtgtctaaactatcaaacgccattatcacactaaatagctactgctaggcaatccttccctcgataaatgtcttggcatcgtttgctttgagcaagaa

SEQ ID NO: 6 - RNA, 4980 nt, Mus musculus
  source 1..4980: mol_type = unassigned RNA
cctcccagtccacctgcagacggcgtataccacgtagcagagggggcatggggggagctggaccttcggttttgcttagtgtttgaccaaaggtgactctgacttagtggactaaggtcatgccctctcttcccccacaatcgataatttaatgtgacttatcagaaagagaacgaattgtttacttttaaaccctggatcccataaagggagagtgcacaaaggcctaaaagccacagatgctcggtgctctcacgcacgcaaggctgccctcaaaccctcagtagagagaagataaacactgtcggaggtaagtgccttcttccagggcctacagtgggagtgaggctgcctgccgaggggagagtgagttgcgggctcaggtgtttaaaagccgccacacagctcctgagcaacactgggcataaaacattccctaattgaatttggtctgccatgattattcattctgttcagaaagcctgagcaaaactggacactttctgaaagcttctttatactccctagagaggggaaggggaaggggaaggctagagacatatggaagacgcagaggctggcttgtatttctatccttggagtggctatggtaagccatgtctcatttaagaatggcctggttatcaatagcatcagggccttggatgaagattctacctgtctgtttaaaatgggggtttgtttgacaggggctcctctttgtgtgtgcttcaccatgccttattcttatttcaaagacacctgatcccaattcttcctcgtgtgtgttccaacttgtactgtcccctcaatgatgctagaaatagctgtccacctgcatagaggtgaaactggtccctaggagtcccgagcttcggcagcccttgattgagagtccatcgagcctttaatgctgagaacctggcggaggattctctctttgcctctggttggtgactaaatggccgatttgattgatgtatggataaggaacttttccagatttctccctttctaaatgggtttccttgaaaagaaaagtgactctgttccctggtgaaaacaagaacattctattttaaaagttctgtgtgctttcaaagatgacaagactcgggagatatgaactgactttctttgtctagactgtactagcctaaaacagccaaatggatacatttcacacaacaacttcacatgacagtaaatgcaggtttgcaatttttttttaaagcatctctgctttttgctttttaagaaatccttttgtggtattgatgggttattttttgcagcaaagtgactccacatagttcccgaagctcccccttcttggtcctgggaagctgtttgcaggatgttcttcgctcctttgaacacgctgaaaatacagattcgtgatcacctggtacttgtgaatccaccacatcttcctaggcgctgtggcgcgcgctgatgcatctgaggcaggttacgctttgccaaggcctcctccttagagaccgcccactcccacttaggaagagagatagatgcctggcattagacagggagcaaacggagtttaacttgtttttcatgcacgggggctcaatggggaaactccatttggaattgtccacagcccctagaaatgtctagaaatcccatcctcttggagaaggggaggacaggacacagaaatcttgttttaataggcagacactaagggacccttgtcactcagcctcccttagggaggacttaggaaataaggcactctgactgctaaatggtaggaggtgtgtgggtctgagctcaagagcatgaaccccttcctgcgctacggaaaccctgctggcatttggacacatcaccctgattaacatccaacaccattggtcttagtcattgctttgttgatgttgtttgttttttttgggagggtgggggtcgggggatgttcatagaaaataacagatgctaccatttttccatccagcatgtgcactttctgttggatattagctagtacaagtgagcttcccagtgttcagtctgaaaattctaaagcccttttaagtctgaagaaaggaaacagacaatgtggcttggaccctagaacgaatggctttggcttttcttctcggtgatacattcattagtaaacaacatttggaagaacaggaaaatattctgctgacagaatttgatcacttggtcaaagaaaacacagaaaaacaaaacaaaacaaatgtttcagtagtgtctaaaaattgttcaaagggccctaataaatgcgaatttatttcttgatataatagcataaagttaactatgctgcatagtacttcagtaattatttgcttaaaaaaaattttttttgacctgaagtctctctctgtcagcaagactgtttcatggtggcaaatacatctgtttgagtcagataggttccagctttcatatttacgaaaagtgtttacaaggaggatttattaaatggggttatccagaaggaggagggaggactttgaaatttttaaaattctcattatttttctcatttccaaagcactgtgaaaaaaattgtgaaaattaataaattcaatgaaacagaggtaaaagtccaacagcagctccctttccacctggtcgagcgctacagtaacatgaacgctgtacagttctccctgaaaagggtccgaatcatatgcactgttttgaaaaccactcctctgttggaaatgaaatcttactttgtttccaagtcaacaacagccaactttttaccgagtcgatgattgcaaggtgaggaccgtggtccaggtgagtgtcgaccatttcccaaagcatgagccatgtgcaaccctttattttaaataatgtcactatgaattagctcttatttactcatttagttagttagttagttagtttaccttttccttgagatgtgtgcttgattcatgacctgcccgaaaggcagagaggttaggggagggttgcgtgtaatatgttggttggttccctcctgttcttcctcctctgtcttttgcacatcaagcactgagaggtcctcagcccctacagcactaatgaatgcctcaccgctcctggtcggccttactacacgtgggaagggggttctagagacctgctgagatggtcagagcatccccgggagggggtgagggatgaaaaagaggtgggatttcagtcctaggtcctccaggcagagaaataaatcagcatagctggtctcttggaaggagtggtggggaaagacaggtcatcacctacacacacacacacacacacacacacacacacacacacacacacacacacacactctcgaggtcccccatcattgcatggccagcacaaaggttttagggtggcgctgggtgtcagcaggagcttcctggtttcacagaggagtgcttgtctaacactaggctgagtgacactcttttcctgcagtctgctggggccacagaggaacaaaggaagccaactattatattttattttttaattttttttagtggacaacaatcctgtcagcaccatagaaaaggaaaggggcgaagaggagccaaagtgggagggaggtcggaggtcacagaatgctgttcctgcggcatctgtcttaaaagggtgtctaaaaggtgtcttaaggtggtctctatgggaatacaggatagaagaatgcgggaggactcgtttcgcttgttacagtttaagcaaatctctagccaaatgttaacaactcagctattctggggatgggggtgagtcaggaaactggcacccaatcttcccccttctcaacatttggggaaaataaaggattaggtaaaactcttttcgttcctctcattctccaccgtgagcagaggttcacagaaccggtgaaaccgtatttaactggagacaagtcaatccagaaggcatttgctcacacagcaaccttcccactagaaagtgaaccttcccgctagaaaaaaaaagtgctaactgtttttatttcattctttcttagttggacccctcacctgctgcctgcagttcttctaaagaatggctgtgcagaatacgacttaaagtttgagcaaagtggggtgtctctaaattcaaagattccacagagaggaggtcctagcccataaagaataatcacaagctgggagtagggggtgtgtgcctttcatcctaatacccaagaggcaggcataggtggatctctgtgattttgagtccagttgggtcgatatagtgagttcaaggccagccagggctacatactaagagaccattgtctcaaaagatagaaaaagaaaagaaaagaagagaagagaaaaggaaaggaagggaagggaaggaaaaggaaaggaaaggaaaggaaaggaaaggaaaggaaaggaaaggaaaggaaaggaaaggaaaggaaaggaaaggaagtcataattttcttgaaccaaagaagtgatcacatttgctagtgatggattggaaaccatgacgaggtgagagtgggtgggatctatcacctaatggagttatttccttgtatgatgtggtttgtaagaagtgtctgcctcttcctggaattcaagccttttaggaaacagatgtaaacaaatttccatcctggtaaaggcctttgtgagccctccttgttattcaagatcccggggctcttccctatgctccagtttcagcgtttggaaccactgaggagtctgtgacagacaacggaggaatgtgaagggaaggttaatacccttgtgcctgacagactttccttagcagagctgtggagtgtgacaatggtgtttgtgtccaaaccatcaaacgccattatcacactaaatagctactgctaggcaatccgtccactccacgcgtgacttgacgtctgttctctccgagcaagaagttctgtcttgtgctagagttctccattgtgtgtaatgtatgaataaaatctggtcc

SEQ ID NO: 7 - protein, 11 aa, human immunodeficiency virus-1
YGRKKRRQRRR

SEQ ID NO: 8 - protein, 12 aa, unidentified
RRQRRTSKLMKR

SEQ ID NO: 9 - protein, 27 aa, unidentified
GWTLNSAGYLLGKINLKALAALAKKIL

SEQ ID NO: 10 - protein, 33 aa, unidentified
KALAWEAKLAKALAKALAKHLAKALAKALKCEA

SEQ ID NO: 11 - protein, 16 aa, unidentified
RQIKIWFQNRRMKWKK

SEQ ID NO: 12 - protein, 9 aa, unidentified
RKKRRQRRR

SEQ ID NO: 13 - protein, 8 aa, unidentified
RKKRRQRR

SEQ ID NO: 14 - protein, 11 aa, unidentified
YARAAARQARA

SEQ ID NO: 15 - protein, 11 aa, unidentified
THRLPRRRRRR

SEQ ID NO: 16 - protein, 11 aa, unidentified
GGRRARRRRRR

SEQ ID NO: 17 - DNA, 19 bp, synthetic construct
  source 1..19: mol_type = other DNA
agaacggcctgatcactca

SEQ ID NO: 18 - DNA, 20 bp, synthetic construct
  source 1..20: mol_type = other DNA
aaaccctggatcccataaag

SEQ ID NO: 19 - DNA, 21 bp, synthetic construct
  source 1..21: mol_type = other DNA
accaaaggtgactctgactta